(19) United States
(12) Patent Application Publication
(10) Pub. No.: US 2018/0078561 A1
(43) Pub. Date: Mar. 22, 2018

(54) ACTIVE METABOLITES OF APILIMOD AND USES THEREOF

(71) Applicant: LAM Therapeutics, Inc., Guilford, CT (US)

(72) Inventors: Paul Beckett, Yorktown Heights, NY (US); Neil Beeharry, Guilford, CT (US); Sean Landrette, Meriden, CT (US); Chris Conrad, Guilford, CT (US); Jonathan M. Rothberg, Guilford, CT (US); Henri Lichenstein, Guilford, CT (US)

(21) Appl. No.: 15/563,115

(22) PCT Filed: Jan. 21, 2016
(86) PCT No.: PCT/US2016/014251

§ 371 Date: Sep. 29, 2017

Related U.S. Application Data
(60) Provisional application No. 62/140,896, filed on Mar. 31, 2015.

(51) Int. Cl. A61K 31/5377

(52) U.S. Cl. CPC A61K 31/5377

(57) ABSTRACT

The present invention relates to compositions comprising one or more active metabolites of apilimod and methods for their use in treating cancer.

### RELATED APPLICATION

This application claims priority to U.S. Patent Application Ser. No. 62/140,896, filed on Mar. 31, 2015, the contents of which are hereby fully incorporated by reference.

### FIELD OF THE INVENTION

The present invention relates to compositions comprising active metabolites of apilimod and methods of using same.

### BACKGROUND OF THE INVENTION

Apilimod, also referred to as STA-5326, hereinafter “apilimod”, is recognized as a potent transcriptional inhibitor of IL-12 and IL-23. See e.g., Wada et al. Blood 109 (2007): 1156-1164. IL-12 and IL-23 are inflammatory cytokines normally produced by immune cells, such as B-cells and macrophages, in response to antigenic stimulation. Autoimmune disorders and other disorders characterized by chronic inflammation are characterized in part by inappropriate production of these cytokines. In immune cells, the selective inhibition of IL-12/IL-23 transcription by apilimod was recently shown to be mediated by apilimod's direct binding to phosphatidylinositol-3-phosphate 5-kinase (PIKfyve). See, e.g., Cai et al. Chemistry and Biol. 20 (2013):912-921. PIKfyve plays a role in Toll-like receptor signaling, which is important in innate immunity.

Based upon its activity as an immunomodulatory agent and a specific inhibitor of IL-12/IL-23, apilimod has been proposed as useful in treating autoimmune and inflammatory diseases and disorders. See e.g., U.S. Pat. Nos. 6,858,606 and 6,660,733 (describing a family of pyrimidine compounds, including apilimod, purportedly useful for treating diseases and disorders characterized by IL-12 or IL-23 overproduction, such as rheumatoid arthritis, sepsis, Crohn's disease, multiple sclerosis, psoriasis, or insulin dependent diabetes mellitus). Similarly, apilimod was suggested to be useful for treating certain cancers based upon its activity to inhibit c-Rel or IL-12/23, particularly in cancers where these cytokines were believed to play a role in promoting aberrant cell proliferation. See e.g., WO 2006/128129 and Baird et al., Frontiers in Oncology 3:1 (2013, respectively).

Each of three clinical trials of apilimod has focused on its potential efficacy in autoimmune and inflammatory diseases. The trials were conducted in patients having psoriasis, rheumatoid arthritis, and Crohn's disease. An open label clinical study in patients with psoriasis concluded that oral administration of apilimod showed immunomodulatory activity supporting the inhibition of IL-12/IL-23 synthesis for the treatment of TH1- and TH17-mediated inflammatory diseases. Wada et al., PLosOne 7:e35069 (April 2012). But the results of controlled trials in rheumatoid arthritis and Crohn's disease did not support the notion that IL-12/IL-23 inhibition by apilimod translates into clinical improvement in either of these indications. In a randomized, double-blind, placebo-controlled Phase II clinical trial of apilimod in patients with rheumatoid arthritis, apilimod failed to alter synovial IL-12 and IL-23 expression. Krauz et al., Arthritis & Rheumatism 64:1750-1755 (2012). The authors concluded that the “results do not support the notion the IL-12/IL-23 inhibition by apilimod is able to induce robust clinical improvement in RA.” Similarly, a randomized, double-blind, placebo-controlled trial of apilimod for treatment of active Crohn's disease concluded that, although well tolerated, apilimod did not demonstrate efficacy over placebo. Sands et al Inflamm Bowel Dis. 2010 July; 16(7):1209-18.

The mammalian target of rapamycin (mTOR) pathway is an important cellular signaling pathway that is involved in multiple physiological functions, including cell growth, cell proliferation, metabolism, protein synthesis, and autophagy (La Plante et al Cell 2012, (149 (2), pp. 274-293). mTOR is a kinase that integrates intracellular and extracellular cues that signal the levels of amino acids, stress, oxygen, energy, and growth factors and regulates the cellular response to these environment cues. mTOR deregulation has been implicated in a wide range of disorders and diseases, including cancer, obesity, diabetes, and neurodegeneration. Certain components of the mTOR pathway have been explored as drug targets for treating some of these diseases. However, therapeutic efficacy has been limited, for example, in the treatment of some cancers, and some mTOR inhibitors have been shown to have an adverse effect on metabolism. The tuberous sclerosis complex tumor suppressor genes, TSC1 and TSC2, are negative regulators of mTOR.

### SUMMARY OF THE INVENTION

The present invention provides methods for treating a disease or disorder in a human subject in need thereof, the method comprising administering an effective amount of a compound of Formula II to the subject:

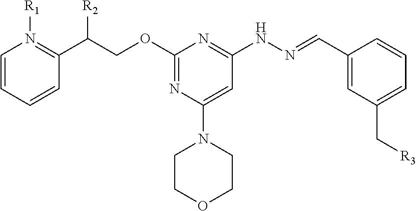

wherein

R₁ is O or absent;

R₂ is H or OH; and

R₃ is H or OH.

In one embodiment, R₂ is OH.

In one embodiment, the effective amount of the compound is the amount effective to inhibit cellular PIKfyve activity in target cells in the subject. In another embodiment, the effective amount is the amount effective to induce vacuolization and disrupts intracellular trafficking in target cells.

In one embodiment, the target cell is a cancer cell. In one embodiment, the cancer cell is a lymphoma cell. In one embodiment, the lymphoma cell is a non-Hodgkins lymphoma cell.

In one embodiment, the disease of disorder is selected from a cancer, a viral infection, a cell proliferative disorder, or Charcot-Marie-Tooth disease (CMT). In one embodiment, the cancer is a lymphoma or melanoma. In one embodiment, the cancer is refractory or resistant to standard therapy. In one embodiment, the cancer is a non-Hodgkins lymphoma.

In one embodiment, the method is a method for treating a lymphoma and the method further comprises administering at least one additional active agent to the subject in a therapeutic regimen comprising a compound of Formula II and the at least one additional active agent. In one embodiment, the at least one additional active agent is selected from ibrutinib, rituximab, doxorubicin, prednisolone, vincristine, velcade, and everolimus, and combinations thereof. In one embodiment, the therapeutic regimen the CHOP regimen.

In one embodiment, the method is a method for treating melanoma and the method further comprises administering at least one additional active agent to the subject in a therapeutic regimen comprising a compound of Formula II and the at least one additional active agent. In one embodiment, the at least one additional active agent is selected from dacarbazine, temozolomide, Nab-paclitaxel, carmustine, cisplatin, carboplatin, or vinblastine.

In one embodiment, the method is a method for treating a viral infection and the method further comprises administering at least one additional active agent to the subject in a therapeutic regimen comprising a compound of Formula II and the at least one additional active agent. In one embodiment, the at least one additional active agent is selected from selected from the group consisting of apilimod, APY0201, and YM-201636.

In accordance with any of the methods described herein, a compound of Formula II may also be administered in combination with a non-therapeutic agent which mitigates one or more side effects associated with the compound of Formula II or increases the bioavailability of a compound of Formula II. In one embodiment, the non-therapeutic agent is selected from the group consisting of ondansetron, granisetron, dolasetron and palonosetron. In another aspect, the non-therapeutic agent is selected from the group consisting of pindolol and risperidone. In another aspect, the non-therapeutic agent is selected from a cytochrome P450 3A (CYP3A) inhibitor. In one embodiment, the CYP3A inhibitor is selected from ritonavir and cobicistat.

In one embodiment, the viral infection is caused by a virus selected from the group consisting of measles, Ebola (EboV), Marburg (MarV), borna disease, and human immunodeficiency virus (HIV), severe acute respiratory system virus (SARS), and middle east respiratory syndrome virus (MERS). In one embodiment, the viral infection is caused by an EboV virus.

In one embodiment, the compound is selected from the group consisting of STA-5864, STA-5944, STA-5908, STA-5919, STA-6035, and STA-6048. In one embodiment, the compound is selected from STA-5944, STA 6048, and STA-5908.

In one embodiment, the compound is in the form a pharmaceutical composition comprising a compound of Formula II and at least one carrier.

In one embodiment, the compound comprises at least 95% or at least 99% enantiomeric excess of the (R)-enantiomer. In one embodiment, the compound comprises at least 95% or at least 99% enantiomeric excess of the (S)-enantiomer.

The invention also provides pharmaceutical compositions comprising a compound of Formula II wherein the compound comprises at least 95% or at least 99% enantiomeric excess of the (R)-enantiomer or the (S)-enantiomer. In one embodiment, the compound is selected from the group consisting of STA-5864, STA-5944, STA-5908, STA-5919, STA-6035, and STA-6048. In one embodiment, the compound is selected from STA-5944, STA 6048, and STA-5908.

### BRIEF DESCRIPTION OF THE DRAWINGS

FIG. 1: TSC2 deficient cells are highly sensitive to apilimod (IC₅₀=20 nM).

FIG. 2A: Sensitivity of cancer cell lines to apilimod (percentage of cell lines with IC₅₀ less than 500 nM).

FIG. 2B: NHL cell lines are particularly sensitive to apilimod (percentage of cell lines with IC₅₀ less than 500 nM).

FIG. 2C: Apilimod's cytotoxic activity is selective for cancer cells over normal cells. Normal lung fibroblasts were insensitive to apilimod-induced cytotoxicity at concentrations as high as 10 micromolar.

FIG. 3: a diffuse large B cell lymphoma, SUDHL-4, exhibited an IC₅₀ of 50 nM.

FIG. 4: Apilimod's cytotoxic activity in NHL cells was a result of increased apoptosis. Apoptotic (Caspase-3/7, middle bar) and necrotic (bis-AAF-R110, right bar) markers in apilimod treated diffuse large B cell lymphoma cells 48 hours after addition of apilimod to the culture media; left bar shows viability marker (GF-AFC).

FIG. 5: Apilimod induces autophagy in a dose-dependent manner.

FIG. 6: Volcano plot of significant captured hits applying CT-689 at 0.1 μM concentration under optimized capture conditions.

FIG. 7: Apilimod binds with high affinity to PIKfyve (Kd=75 pM).

FIG. 8: IL-23A expression is not a statistically significant predictor of sensitivity in Non-Hodgkin's B cell lymphoma. Shown are apilimod sensitive NHL cell lines (bottom, dark) and insensitive (top, light).

FIG. 9: Apilimod inhibits the growth of SU-DHL-6 DLBCL xenograft tumors; Top graph shows tumor size for vehicle alone (saline, diamond, light grey solid lines) QD ×5, 2 days off, QD ×5 i.v.; 0.5% methylcellulose (triangle, solid dark grey lines) QD ×5, 2 days off, QD ×5 p.o.; apilimod dimesylate (square, dashed lines) 67.5 mg/kg (47 mg/kg free base) QD ×5 i.v., 2 days off, QD ×5; apilimod free base (square, light grey solid lines) 150 mg/kg QD ×5, 2 days off, QD ×5 p.o; apilimod free base (cross, solid lines) 75 mg/kg BID ×5, 2 days off, BID ×5 p.o. Bottom graph shows body weight versus days post tumor inoculation.

FIG. 10: Antitumor activity of apilimod in combination with ibrutinib on DLBCL tumors in vivo; Top graph shows tumor size for vehicle (diamond, light grey solid lines) QD ×5, 2 days off, QD ×5 p.o. +i.v.; ibrutinib (triangle, solid dark grey lines) 10 mg/kg QD ×12 i.v.; apilimod free base (square, dashed lines) 75 mg/kg QD ×5, 2 days off, QD ×5 p.o.; ibrutinib (cross, solid dark line) 20 mg/kg QD ×12 i.v.; apilimod free base 75 mg/kg QD ×5, 2 days off, QD ×5 p.o. +ibrutinib 10 mg/kg QD ×12 i.v. (square, solid light grey lines); apilimod free base 75 mg/kg QD ×5, 2 days off, QD ×5 p.o. +ibrutinib 10 mg/kg QD ×12 i.v. (circle, solid medium grey lines). Bottom graph shows body weight versus days after tumor inoculation.

FIG. 11: Screening SU-DHL-4 cells with a manually curated library of 93 drugs with and without apilimod (10 nM) identified ibrutinib as a drug that when combined with apilimod exerts synergistic activity.

FIG. 12: Apilimod induces vacuolization of a representative cancer cell line. Left: Untreated cell. Right: Live cells treated with 500 nM apilimod 24 h.

FIG. 13: Screening Yulac614 melanoma cells with a library of 500 unapproved drugs with and without vemurafenib (6 μM) identified apilimod as a drug that when combined with vermurafenib exerts synergistic activity.

FIG. 14: 10 point concentration response curve of vemurafenib (58.6-30,000 nM) alone (black line) or with apilimod (500 nM) (grey line).

FIG. 15: IC50 values in vemurafenib-resistant cell lines treated with the vemurafenib alone (grey bars) or the combination of vemurafenib and apilimod (black bars).

FIG. 16: IC50 values in vemurafenib-resistant cell lines treated with the vemurafenib alone (grey bars) or the combination of vemurafenib and apilimod (black bars).

FIG. 17: Representative K_{d} curves of A) STA-5908, B) STA-5944 and C) STA-6048 tested against PIKfyve.

FIG. 18: PK Profile of PK of Apilimod (STA 5326) and Metabolites in Healthy Human Volunteers (2×10⁵ mg, 10 hours apart).

FIG. 19: Mean Plasma Apilimod (API) or Plasma Total Apilimod Effect Concentration (TEAC) in ng/mL versus Time. Apilimod free base was administered in two 105 mg doses, 10 hours apart.

### DETAILED DESCRIPTION OF THE INVENTION

The present invention provides compounds which are active metabolites of apilimod, related compositions, and methods related to their use for treating certain diseases and disorders. In one aspect, the compounds are described by Formula II, infra. In various aspects of the invention, methods are provided for treating a cell proliferative disease, a cancer, a viral infection, or Charcot-Marie-Tooth disease (CMT) in a subject, preferably a human subject, in need of such treatment, administering an effective amount of a compound of Formula II or a pharmaceutical composition comprising same, to the subject.

Applicant discovered that apilimod is a highly cytotoxic agent in TSC null cells in which the mTOR pathway is constitutively active. The mTOR pathway is activated in a number of cancers, and in further screening of over 100 cancer cell lines apilimod showed anti-proliferative activity in cell lines from diverse cancers. Surprisingly, apilimod's cytotoxic activity against this range of cancer cells of both lymphoid and non-lymphoid origin, is not clearly related to, or predictable from, apilimod's known immunomodulatory and IL-12/23 inhibitory activity. Among the apilimod sensitive cancer cell lines, B-cell lymphomas were the most sensitive. But, unexpectedly, the differential sensitivity of B cell lymphomas to apilimod did not correlate with c-Rel expression, IL-12 expression, or IL-23 expression in these cells. This was surprising because earlier work had suggested apilimod would be useful against cancers where c-Rel and/or IL-12/23 expression were critical in promoting aberrant cell proliferation. Instead, Applicant demonstrated that apilimod's cytotoxic activity in cancer cells was due to an inhibition of intracellular trafficking and a corresponding increase in apoptosis. This activity was not predicted based upon apilimod's immunomodulatory activity via its inhibition of IL-12/23 production.

Applicant also identified PIKfyve as the sole high affinity binding target (Kd=75 pM) of apilimod in a screen of over 450 kinases. PIKfyve is a phosphoinositide kinase (PIK) that contains a FYVE-type zinc finger domain, which binds phosphatidylinositol 3-phosphate (PI3P). PIKfyve phosphorylates PUP to produce PI(3,5)P₂, which is involved in cellular processes including membrane trafficking and cytoskeletal reorganization. As described in more detail infra, the inhibition of PIKfyve by a composition of the invention is useful in treating not only cancer, but also Charcot-Marie-Tooth disease and certain viral infections, especially those caused by a virus selected from the group consisting of measles, Ebola (EboV), Marburg (MarV), borna disease, and human immunodeficiency virus (HIV), severe acute respiratory system virus (SARS), and middle east respiratory syndrome virus (MERS).

The present invention extends the Applicant's earlier work by identifying and providing active metabolites of apilimod which are at least as effective as apilimod itself in binding to and inhibiting PIKfyve kinase and which further exhibit similar cellular effects as apilimod, e.g., on intracellular trafficking and anti-proliferative activity. In addition, the invention further provides compositions comprising one or more enantiomers of an active metabolite of apilimod. Preferably, a compound of Formula II comprises at least 95% or at least 99% enantiomeric excess of the (R)- or (S)-enantiomer. In certain embodiments, the enantiomer of a compound of Formula II has increased biological activity compared to apilimod itself. In certain embodiments, the biological activity is measured as inhibition of PIKfyve kinase activity, inhibition of intracellular trafficking, anti-viral activity, cytotoxicity, or anti-proliferative activity.

Thus, the present invention provides methods for treating a disease or disorder selected from the group consisting of cancer, an mTOR related disease or disorder, CMT, and a viral infection by administering to a subject in need thereof a composition comprising one or more active metabolites of apilimod. In one embodiment, the metabolite is in a racemically pure form.

In one embodiment, the active metabolites of apilimod described herein are useful for treating cancer. In one embodiment, the cancer is a B cell lymphoma. In one embodiment the cancer is a B cell lymphoma that is resistant or refractory to standard chemotherapy regimens. In another embodiment, the cancer is a melanoma. In one embodiment the cancer is a melanoma that is resistant or refractory to standard chemotherapy regimens. In addition, the present invention provides novel therapeutic approaches to cancer treatment based upon combination therapy utilizing active metabolites of apilimod and at least one additional therapeutic agent. The combination therapies described herein exploit the unique cytotoxic activity of apilimod which is shown to provide a synergistic effect when combined with other therapeutic agents, including for example, anti-cancer agents. The methods of treating cancer with a composition of the invention are described in more detail infra.

Another aspect of the invention provides methods for the treatment of a viral infection in a subject by administering to the subject a therapeutically effective amount of a composition described herein. In one embodiment, the viral infection is caused by a virus selected from the group consisting of measles, Ebola (EboV), Marburg (MarV), borna disease, and human immunodeficiency virus (HIV), severe acute respiratory system virus (SARS), and middle east respiratory syndrome virus (MERS). In one embodiment, the viral infection is caused by EboV or MarV. The methods of treating viral infections with a composition of the invention are described in more detail, infra.

The compositions of the present invention comprise one or more active metabolites of apilimod. As used herein, the term “an active metabolite of apilimod” may refer to one of the active metabolites in its free base form, as set forth in Table 1, or may encompass pharmaceutically acceptable salts, solvates, clathrates, hydrates, polymorphs, prodrugs, analogs or derivatives thereof, as described below. In one embodiment, an active metabolite of apilimod is in an enantiomerically pure form, that is, it consists of a single enantiomer (R or S) present in about greater than 99% enantiomeric excess compared to the other enantiomer.

The structure of the parent compound, apilimod is shown in Formula I:

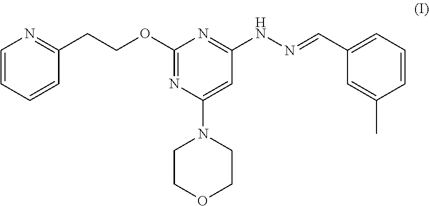 [SMILES: Cc1cccc(/C=N/Nc2cc(N3CCOCC3)nc(OCCc3ccccn3)n2)c1]

The chemical name of apilimod is 2-[2-Pyridin-2-yl)-ethoxy]-4-N′-(3-methyl-benzilidene)-hydrazino]-6-(morpholin-4-yl)-pyrimidine (IUPAC name: (E)-4-(6-(2-(3-methylbenzylidene)hydrazinyl)-2-(2-(pyridin-2-yl)ethoxy)pyrimidin-4-yl)morpholine), and the CAS number is 541550-19-0.

An active metabolite of apilimod, as provided herein, encompasses compounds described by Formula II:

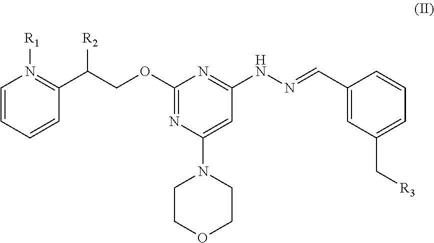

wherein
R₁ is O or absent;

### R₂ is H or OH; and

### R₃ is H or OH.

In some embodiments, the compounds of Formula (I) are those in which R₁ is O.
For example, R₁ is O and R₂ is OH.
For example, R₁ is O and R₂ is H.
For example, R₁ id O, R₂ is OH and R₃ is H.
In some embodiments, the compounds of Formula (II) are those in which R₁ is absent.
For example, R₁ is absent and R₂ is OH.
For example, R₁ is absent, R₂ is H and R₃ is OH.

In one embodiment, a compound of Formula (II) is in an enantiomerically pure form, that is, it consists of a single enantiomer (R or S) present in about greater than 99% enantiomeric excess compared to the other enantiomer. An enantiomer refers to a compound which has at least one chiral center and therefore may exist as either an (R) or (S) enantiomer at each chiral center. Typically, such compounds comprise about the same amount of each enantiomer, e.g., about 50% of each enantiomer. The active metabolites of apilimod described herein each have a single chiral center at the carbon atom bonded to R₂ (circled in Formula II). In one embodiment, the enantiomer present in >99% enantiomeric excess will be the (R)-enantiomer of compounds of Formula (II), wherein R₂ is OH. In one embodiment the enantiomer present in >99% enantiomeric excess is the (S)-enantiomer of a compound of Formula (II), wherein R₂ is OH. It is to be understood that the R and S stereochemistry of the individual enantiomers is determined by the substitution pattern of chemical functional groups attached to the carbon atom bonded to R₂, as would be easily recognized by a skilled person in the arts.

Specific examples of compounds of Formula (II) include Compounds 1-6 in the table below:

TABLE 1
Active metabolites of apilimod

| Compound | Formula | Name |
|---|---|---|
| Compound 1 | 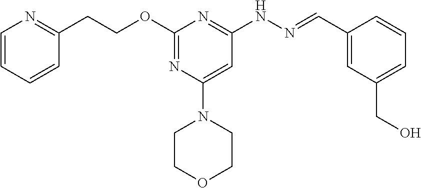 [SMILES: OCc1cccc(/C=N/Nc2cc(N3CCOCC3)nc(OCCc3ccccn3)n2)c1] | STA-5864 |
| Compound 2 | 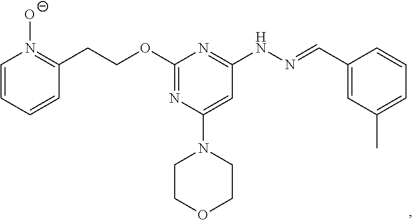 | STA-5944 |
| Compound 3 | 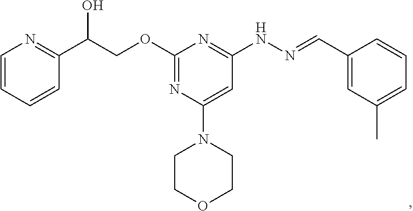 [SMILES: Cc1cccc(/C=N/Nc2cc(N3CCOCC3)nc(OCC(O)c3ccccn3)n2)c1] | STA-5908 |
| Compound 4 | 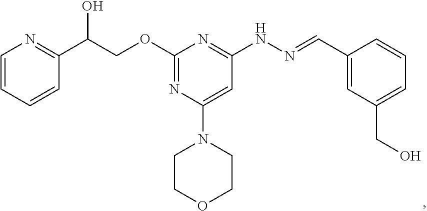 [SMILES: OCc1cccc(/C=N/Nc2cc(N3CCOCC3)nc(OCC(O)c3ccccn3)n2)c1] | STA-5919 |
| Compound 5 | 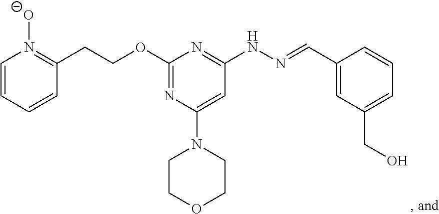 | STA-6035 |
| Compound 6 | 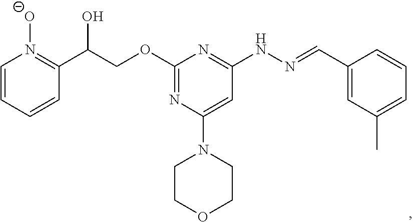 | STA-6048 |

Apilimod metabolites can be prepared analogous to synthetic routes used in the synthesis of the parent compound apilimod, for example, according to the methods described in U.S. Pat. Nos. 7,923,557, and 7,863,270, and WO 2006/128129.

Compounds 1-6 each contain a stereocenter and therefore may comprise a mixture of (R) and (S)-enantiomers. The ratio of both enantiomers can range from about 1:99 to about 99:1. In certain embodiments, any of compounds 1-6 may also be prepared as individual enantiomers with >99% enantiomeric excess. For example, the (R)-enantiomer of compounds of Formula (II), wherein R₂ is OH, is present in >99% enantiomeric excess. In another example, the (S)-enantiomer of compounds of Formula (II), wherein R₂ is OH, is present in >99% enantiomeric excess. It is to be understood that enantiomeric excess (ee) is a measurement of purity used for chiral substances. It reflects the degree to which a sample contains one enantiomer in greater amounts than the other. A racemic mixture has an ee of 0%, while a single completely pure enantiomer has an ee of 100%. For example, a compound mixture with 70% of one enantiomer and 30% of the other has an ee of 40%. Enantiomeric excess is chemically defined as the absolute difference between the mole fraction of each enantiomer present in the compound mixture. In one embodiment, the (R)-enantiomer of a compound of Formula (II), wherein R₂ is OH, is be present in 10-, 20, 30, 40, 50, 60, 70, 80, 90, 95, or >95% enantiomeric excess within the compound mixture. In another embodiment, the (S)-enantiomer of a compound of Formula (II), wherein R₂ is OH, is be present in 10-, 20, 30, 40, 50, 60, 70, 80, 90, 95, or >95% enantiomeric excess within the compound mixture.

In one embodiment, an active metabolite of apilimod for use in the compositions and methods of the invention is selected from the group consisting of STA-5908, STA-5944, and STA-6048, either in its free base form, or a pharmaceutically acceptable salt, solvate, clathrate, hydrate, polymorph, prodrug, analog or derivative thereof. In one embodiment, the active metabolite of apilimod is present in enantiomeric excess of at least 90%, at least 95%, or at least 99%.

In one embodiment, the active metabolite of apilimod is the free base or dimesylate salt form. The apilimod metabolite dimesylate salt can be highly water soluble (>25 mg/mL) and may exhibit moderate permeability (>70% in rats). Compounds 1-6 were identified as apilimod metabolites in rat and human microsomal and hepatocyte stability studies. Human, rat, rabbit and dog studies showed a qualitatively similar metabolic profile. Tₘₐₓ generally occurred within 1 or 2 hours after the oral dose, consistent with the rapid elimination of this compound from the circulation. Reaction phenotyping studies indicated that CYP3A4 and to a lesser extent CYP1A2 and/or CYP2D6, contribute to metabolism. The primary metabolites are short-lived in circulation. Both apilimod free base and the dimesylate salt are highly bound (>99%) to rat, dog and human plasma proteins.

The present invention provides compositions that are preferably pharmaceutically acceptable compositions suitable for use in a mammal, preferably a human. In this context, the compositions may further comprise at least one pharmaceutically acceptable excipient or carrier, wherein the amount is effective for the treatment of a disease or disorder.

As used herein, the term “pharmaceutically acceptable salt,” is a salt formed from, for example, an acid and a basic group of an apilimod composition. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, besylate, gentisinate, fumarate, gluconate, glucaronate, saccharate, formate, benzoate, glutamate, methanesulfonate, ethanesulfonate, benzenesulfonate, p-toluenesulfonate, and pamoate (e.g., 1,1′-methylene-bis-(2-hydroxy-3-naphthoate)) salts. In a preferred embodiment, the salt of apilimod comprises methanesulfonate.

The term “pharmaceutically acceptable salt” also refers to a salt prepared from an apilimod composition having an acidic functional group, such as a carboxylic acid functional group, and a pharmaceutically acceptable inorganic or organic base.

The term “pharmaceutically acceptable salt” also refers to a salt prepared from an apilimod composition having a basic functional group, such as an amino functional group, and a pharmaceutically acceptable inorganic or organic acid.

The salts of the compounds described herein can be synthesized from the parent compound by conventional chemical methods such as methods described in Pharmaceutical Salts: Properties, Selection, and Use, P. Hemrich Stalil (Editor), Camille G. Wermuth (Editor), ISBN: 3-90639-026-8, August 2002. Generally, such salts can be prepared by reacting the parent compound with the appropriate acid in water or in an organic solvent, or in a mixture of the two.

One salt form of a compound described herein can be converted to the free base and optionally to another salt form by methods well known to the skilled person. For example, the free base can be formed by passing the salt solution through a column containing an amine stationary phase (e.g. a Strata-NH₂ column). Alternatively, a solution of the salt in water can be treated with sodium bicarbonate to decompose the salt and precipitate out the free base. The free base may then be combined with another acid using routine methods.

As used herein, the term “polymorph” means solid crystalline forms of a compound of the present invention (e.g., 2-[2-Pyridin-2-yl)-ethoxy]-4-N′-(3-methyl-benzilidene)-hydrazino]-6-(morpholin-4-yl)-pyrimidine) or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “hydrate” means a compound of the present invention (e.g., 2-[2-Pyridin-2-yl)-ethoxy]-4-N′-(3-methyl-benzilidene)-hydrazino]-6-(morpholin-4-yl)-pyrimidine) or a salt thereof, which further includes a stoichiometric or non-stoichiometric amount of water bound by non-covalent intermolecular forces.

As used herein, the term “clathrate” means a compound of the present invention (e.g., 2-[2-Pyridin-2-yl)-ethoxy]-4-N′-(3-methyl-benzilidene)-hydrazino]-6-(morpholin-4-yl)-pyrimidine) or a salt thereof in the form of a crystal lattice that contains spaces (e.g., channels) that have a guest molecule (e.g., a solvent or water) trapped within.

As used herein, the term “prodrug” means a derivative of a compound described herein (e.g., 2-[2-Pyridin-2-yl)-ethoxy]-4-N′-(3-methyl-benzilidene)-hydrazino]-6-(morpholin-4-yl)-pyrimidine) that can hydrolyze, oxidize, or otherwise react under biological conditions (in vitro or in vivo) to provide a compound of the invention. Prodrugs may only become active upon such reaction under biological conditions, or they may have activity in their unreacted forms. Examples of prodrugs contemplated in this invention include, but are not limited to, analogs or derivatives of a compound described herein (e.g., 2-[2-Pyridin-2-yl)-ethoxy]-4-N′-(3-methyl-benzilidene)-hydrazino]-6-(morpholin-4-yl)-pyrimidine) that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds of any one of the formulae disclosed herein that comprise —NO, —NO₂, —ONO, or —ONO₂ moieties. Prodrugs can typically be prepared using well-known methods, such as those described by Burger's Medicinal Chemistry and Drug Discovery (1995) 172-178, 949-982 (Manfred E. Wolff ed., 5th ed).

As used herein, the term “solvate” or “pharmaceutically acceptable solvate,” is a solvate formed from the association of one or more solvent molecules to one of the compounds disclosed herein (e.g., 2-[2-Pyridin-2-yl)-ethoxy]-4-N′-(3-methyl-benzilidene)-hydrazino]-6-(morpholin-4-yl)-pyrimidine). The term solvate includes hydrates (e.g., hemi-hydrate, mono-hydrate, dihydrate, trihydrate, tetrahydrate, and the like).

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound. As used herein, the term “derivative” refers to compounds that have a common core structure, and are substituted with various groups as described herein.

In the context of the methods described herein, the amount of a composition administered to the subject is a therapeutically effective amount. The term “therapeutically effective amount” refers to an amount sufficient to treat, ameliorate a symptom of, reduce the severity of, or reduce the duration of the disease or disorder being treated, or enhance or improve the therapeutic effect of another therapy, or sufficient to exhibit a detectable therapeutic effect in the subject. In one embodiment, the therapeutically effective amount of an apilimod composition is the amount effective to inhibit PIKfyve kinase activity.

An effective amount of a composition described herein can range from about 0.001 mg/kg to about 1000 mg/kg, about 0.01 mg/kg to about 100 mg/kg, about 10 mg/kg to about 250 mg/kg, about 0.1 mg/kg to about 15 mg/kg; or any range in which the low end of the range is any amount between 0.001 mg/kg and 900 mg/kg and the upper end of the range is any amount between 0.1 mg/kg and 1000 mg/kg (e.g., 0.005 mg/kg and 200 mg/kg, 0.5 mg/kg and 20 mg/kg). Effective doses will also vary, as recognized by those skilled in the art, depending on the diseases treated, route of administration, excipient usage, and the possibility of co-usage with other therapeutic treatments such as use of other agents. See, e.g., U.S. Pat. No. 7,863,270, incorporated herein by reference.

In more specific aspects, a composition as described herein is administered at a dosage regimen of 30-1000 mg/day (e.g., 30, 35, 40, 45, 50, 55, 60, 65, 70, 75, 80, 85, 90, 95, 100, 125, 150, 175, 200, 225, 250, 275, or 300 mg/day) for at least 1 week (e.g., 1, 2, 3, 4, 5, 6, 7, 8, 9, 10, 11, 12, 36, 48, or more weeks). Preferably, the composition is administered at a dosage regimen of 100-1000 mg/day for 4 or 16 weeks. Alternatively or subsequently, the composition is administered at a dosage regimen of 100 mg-300 mg twice a day for 8 weeks, or optionally, for 52 weeks. Alternatively or subsequently, the composition is administered at a dosage regimen of 50 mg-1000 mg twice a day for 8 weeks, or optionally, for 52 weeks.

An effective amount of the composition can be administered once daily, from two to five times daily, up to two times or up to three times daily, or up to eight times daily. In one embodiment, the composition is administered thrice daily, twice daily, once daily, fourteen days on (four times daily, thrice daily or twice daily, or once daily) and 7 days off in a 3-week cycle, up to five or seven days on (four times daily, thrice daily or twice daily, or once daily) and 14-16 days off in 3 week cycle, or once every two days, or once a week, or once every 2 weeks, or once every 3 weeks.

In accordance with the methods described herein, a “subject in need of” is a subject having a disease, disorder or condition, or a subject having an increased risk of developing a disease, disorder or condition relative to the population at large. The subject in need thereof can be one that is “non-responsive” or “refractory” to a currently available therapy for the disease or disorder, for example cancer. In this context, the terms “non-responsive” and “refractory” refer to the subject's response to therapy as not clinically adequate to relieve one or more symptoms associated with the disease or disorder. In one aspect of the methods described here, the subject in need thereof is a subject having cancer whose cancer is refractory to standard therapy or whose cancer has recurred following standard treatment.

A “subject” includes a mammal. The mammal can be e.g., any mammal, e.g., a human, primate, vertebrate, bird, mouse, rat, fowl, dog, cat, cow, horse, goat, camel, sheep or a pig. Preferably, the mammal is a human. The term “patient” refers to a human subject.

The present invention also provides a monotherapy for the treatment of a disease, disorder or condition as described herein. As used herein, “monotherapy” refers to the administration of a single active or therapeutic compound to a subject in need thereof.

As used herein, “treatment”, “treating” or “treat” describes the management and care of a patient for the purpose of combating a disease, condition, or disorder and includes the administration of an apilimod composition to alleviate the symptoms or complications of a disease, condition or disorder, or to eliminate the disease, condition or disorder.

As used herein, “prevention”, “preventing” or “prevent” describes reducing or eliminating the onset of the symptoms or complications of the disease, condition or disorder and includes the administration of an apilimod composition to reduce the onset, development or recurrence of symptoms of the disease, condition or disorder.

Treating a disorder, disease or condition according to the methods described herein can result in a decrease in the mortality rate of a population of treated subjects in comparison to a population receiving carrier alone. Treating a disorder, disease or condition according to the methods described herein can result in a decrease in the mortality rate of a population of treated subjects in comparison to an untreated population. Treating a disorder, disease or condition according to the methods described herein can result in a decrease in the mortality rate of a population of treated subjects in comparison to a population receiving monotherapy with a drug other than a composition of the invention. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating a disorder, disease or condition according to the methods described herein can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating a disorder, disease or condition according to the methods described herein can result in increase in average survival time of a population of treated subjects in comparison to a population receiving monotherapy with a drug that is not a composition described herein. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

### Methods of Treating Cancer

The present invention provides methods for the treatment of cancer in a subject in need thereof by administering to the subject a therapeutically effective amount of a composition comprising one or more active metabolites of apilimod as described herein, said composition comprising one or more active metabolites of apilimod in the free base form, or a pharmaceutically acceptable salt, solvate, clathrate, hydrate, polymorph, prodrug, analog or derivative thereof. In one embodiment, the composition comprises at least one active metabolite of apilimod in its free base form, or a dimesylate salt thereof.

In one embodiment, the cancer is brain cancer, glioma, sarcoma, breast cancer, lung cancer, non-small-cell lung cancer, mesothelioma, appendiceal cancer, genitourinary cancers, renal cell carcinoma, prostate cancer, bladder cancer, testicular cancer, penile cancer, cervical cancer, ovarian cancer, von Hippel Lindau disease, head and neck cancer, gastrointestinal cancer, hepatocellular carcinoma, gallbladder cancer, esophageal cancer, gastric cancer, colorectal cancer, pancreatic cancer, neuroendocrine tumors, thyroid tumor, pituitary tumor, adrenal tumor, hematological malignancy, or leukemia.

In one embodiment the cancer is a lymphoma. In one embodiment, the lymphoma is a B cell lymphoma. In one embodiment, the B cell lymphoma is selected from the group consisting of a Hodgkin's B cell lymphoma and a non-Hodgkin's B cell lymphoma. In one embodiment, the B cell lymphoma is a non-Hodgkin's B cell lymphoma selected from the group consisting of DLBCL, follicular lymphoma, marginal zone lymphoma (MZL) or mucosa associated lymphatic tissue lymphoma (MALT), small cell lymphocytic lymphoma (overlaps with chronic lymphocytic leukemia) and mantle cell lymphoma. In one embodiment, the B cell lymphoma is a non-Hodgkin's B cell lymphoma selected from the group consisting of Burkitt's lymphoma, Primary mediastinal (thymic) large B-cell lymphoma, Lymphoplasmacytic lymphoma, which may manifest as Waldenström macroglobulinemia, Nodal marginal zone B cell lymphoma (NMZL), Splenic marginal zone lymphoma (SMZL), Intravascular large B-cell lymphoma, Primary effusion lymphoma, Lymphomatoid granulomatosis, T cell/histiocyte-rich large B-cell lymphoma, Primary central nervous system lymphoma, Primary cutaneous diffuse large B-cell lymphoma, leg type (Primary cutaneous DLBCL, leg type), EBV positive diffuse large B-cell lymphoma of the elderly, Diffuse large B-cell lymphoma associated with inflammation, Intravascular large B-cell lymphoma, ALK-positive large B-cell lymphoma, and Plasmablastic lymphoma.

In one embodiment, the human cancer patient in need of treatment with an apilimod composition of the invention is one whose cancer is refractory to a standard chemotherapy regimen. In one embodiment, the human cancer patient in need of treatment with an apilimod composition is one whose cancer has recurred following treatment with a standard chemotherapy regimen. In one embodiment, the cancer is a lymphoma. In one embodiment, the cancer is a B cell lymphoma. In one embodiment, the B cell lymphoma is a non-Hodgkin's B cell lymphoma. In one embodiment, the non-Hodgkin's B cell lymphoma is selected from a diffuse large B cell lymphoma (DLBCL), a Burkitt's lymphoma, a mediastinal B cell lymphoma, a mantle cell lymphoma, and a follicular lymphoma. In one embodiment, the non-Hodgkin's B cell lymphoma is DLBCL. In one embodiment, the DLBCL is the GCB subtype.

In one embodiment, the standard chemotherapy regimen comprises one or more therapeutic agents selected from the group consisting of ibrutinib, rituximab, doxorubicin, prednisolone, vincristine, velcade, cyclophosphamide, dexamethasone and everolimus. In one embodiment, the standard chemotherapy regimen is selected from CHOP, (cyclophosphamide, hydroxydaunorubicin, Oncovin™ (vincristine), and prednisone or prednisolone), COOP (cyclophosphamide, vincristine sulfate, procarbazine hydrochloride, prednisone), CVP (cyclophosphamide, vincristine sulfate, prednisone), EPOCH (etoposide, prednisone, vincristine sulfate, cyclophosphamide, doxorubicin hydrochloride), Hyper-CVAD (cyclophosphamide, vincristine sulfate, doxorubicin hydrochloride, dexamethasone), ICE (ifosfamide, carboplatin, etoposide), R-CHOP (rituximab, cyclophosphamide, vincristine sulfate, procarbazine hydrochloride, prednisone, and R-CVP (rituximab, cyclophosphamide, vincristine sulfate, prednisone).

In one embodiment, the method is a method of treating a lymphoma using a combination therapy comprising an apilimod composition and a chemotherapy regimen for the treatment of the lymphoma. In one embodiment, the chemotherapy regimen is the CHOP regimen. In another embodiment, the chemotherapy regimen is selected from COOP, CVP, EPOCH, Hyper-CVAD, ICE, R-CHOP, and R-CVP.

In one embodiment, the cancer is a melanoma and the “subject in need of” is a subject having melanoma. In one aspect, the subject is a human patient having malignant melanoma or late-stage melanoma. In this context, “stage” refers to the clinical stage of the cancer. For example, stage 0 to 2 melanoma or stage 3 or stage 4 melanoma. In one embodiment, the subject is a human patient having stage 3 or stage 4 melanoma. The subject in need thereof can be one that is “non-responsive” or “refractory” to a currently available therapy, for example the subject's cancer may be resistant or refractor to treatment with vemurafenib. In this context, the terms “non-responsive” and “refractory” refer to the subject's response to therapy as not clinically significant according to the definition for a clinical response in standard medical practice.

In one embodiment, the administration of a composition of the invention leads to the elimination of a symptom or complication of the disease or disorder being treated, however, elimination is not required. In one embodiment, the severity of the symptom is decreased. In the context of cancer, such symptoms may include clinical markers of severity or progression including the degree to which a tumor secrets growth factors, degrades the extracellular matrix, becomes vascularized, loses adhesion to juxtaposed tissues, or metastasizes, as well as the number of metastases.

Treating cancer according to the methods described herein can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer according to the methods described herein can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer according to the methods described herein can result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer according to the methods described herein can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer according to the methods described herein can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time. In one embodiment, after treatment the tumor growth rate may be about zero and is determined to maintain the same size, e.g., has stopped growing.

### Combination Therapy for Treating Cancer

The present invention also provides methods comprising combination therapy. As used herein, “combination therapy” or “co-therapy” includes the administration of a therapeutically effective amount of a compound described herein with at least one additional active agent, as part of a specific treatment regimen intended to provide a beneficial effect from the co-action of the compound of the invention and the additional active agent. “Combination therapy” is not intended to encompass the administration of two or more therapeutic compounds as part of separate monotherapy regimens that incidentally and arbitrarily result in a beneficial effect that was not intended or predicted.

In one embodiment, the at least one additional active agent is selected from the group consisting of an alkylating agent, an intercalating agent, a tubulin binding agent, a corticosteroid, and combinations thereof. In one embodiment, the at least one additional active agent is a therapeutic agent selected from the group consisting of ibrutinib, rituximab, doxorubicin, prednisolone, vincristine, velcade, and everolimus, and combinations thereof. In one embodiment, the at least one additional active agent is a therapeutic agent selected from cyclophosphamide, hydroxydaunorubicin (also referred to as doxorubicin or Adriamycin™) vincristine (also referred to as Oncovin™), prednisone, prednisolone, and combinations thereof. In one embodiment, the at least one additional active agent is a non-therapeutic agent selected to ameliorate one or more side effects of the apilimod composition. In one embodiment, the non-therapeutic agent is selected from the group consisting of ondansetron, granisetron, dolasetron and palonosetron. In one embodiment, the non-therapeutic agent is selected from the group consisting of pindolol and risperidone.

In one embodiment, the method is a method of treating cancer using a combination therapy comprising a compound of Formula II and at least one additional active agent in a therapeutic regimen comprising a compound of Formula II and the at least one additional active agent. In one embodiment, the chemotherapy regimen is the CHOP regimen. CHOP refers to a regimen generally used in the treatment of non-Hodgkin's lymphoma consisting of the following active agents: (C)yclophosphamide, an alkylating agent which damages DNA by binding to it and causing the formation of cross-links; (H)ydroxydaunorubicin (also called doxorubicin or Adriamycin), an intercalating agent which damages DNA by inserting itself between DNA bases; (O)ncovin (vincristine), which prevents cells from duplicating by binding to the protein tubulin; and (P)rednisone or (P)rednisolone, which are corticosteroids. In another embodiment, the chemotherapy regimen is selected from COOP (cyclophosphamide, vincristine sulfate, procarbazine hydrochloride, prednisone), CVP (cyclophosphamide, vincristine sulfate, prednisone), EPOCH (etoposide, prednisone, vincristine sulfate, cyclophosphamide, doxorubicin hydrochloride), Hyper-CVAD (cyclophosphamide, vincristine sulfate, doxorubicin hydrochloride, dexamethasone), ICE (ifosfamide, carboplatin, etoposide), R-CHOP (rituximab, cyclophosphamide, vincristine sulfate, procarbazine hydrochloride, prednisone, and R-CVP (rituximab, cyclophosphamide, vincristine sulfate, prednisone).

In one embodiment, the method is a method for treating melanoma and the method further comprises administering at least one additional active agent to the subject in a therapeutic regimen comprising a compound of Formula II and the at least one additional active agent. In one embodiment, the at least one additional active agent is selected from dacarbazine, temozolomide, Nab-paclitaxel, carmustine, cisplatin, carboplatin, or vinblastine.

In accordance with the methods for combination therapy described herein, the at least one additional active agent may be a therapeutic agent, for example an anti-cancer agent or a cancer chemotherapeutic agent, or a non-therapeutic agent, and combinations thereof. With respect to therapeutic agents, the beneficial effect of the combination includes, but is not limited to, pharmacokinetic or pharmacodynamic co-action resulting from the combination of therapeutically active compounds. With respect to non-therapeutic agents, the beneficial effect of the combination may relate to the mitigation of a toxicity, side effect, or adverse event associated with a therapeutically active agent in the combination.

In one embodiment, the at least one additional agent is a non-therapeutic agent which mitigates one or more side effects of an apilimod composition, the one or more side effects selected from any of nausea, vomiting, headache, dizziness, lightheadedness, drowsiness and stress. In one aspect of this embodiment, the non-therapeutic agent is an antagonist of a serotonin receptor, also known as 5-hydroxytryptamine receptors or 5-HT receptors. In one aspect, the non-therapeutic agent is an antagonist of a 5-HT₃ or 5-HT₁ₐ receptor. In one aspect, the non-therapeutic agent is selected from the group consisting of ondansetron, granisetron, dolasetron and palonosetron. In another aspect, the non-therapeutic agent is selected from the group consisting of pindolol and risperidone.

In one embodiment, the at least one additional agent is a therapeutic agent. In one embodiment, the therapeutic agent is an anti-cancer agent. In one embodiment, the anti-cancer agent is ibrutinib. In one embodiment, an apilimod composition is administered along with ibrutinib in a single dosage form or in separate dosage forms. In one embodiment, the dosage form is an oral dosage form. In another embodiment, the dosage form is suitable for intravenous administration.

In one embodiment, the anti-cancer agent is a drug that is approved for use in treating lymphoma. Non-limiting examples of such drugs include abitrexate (methotrexate), adcetris (brentuximab vedotin), ambochlorin (chlorambucil), amboclorin (chloramucil), arranon (nelarabine), becenum (carmustine), beleodaq (belinostat), belinostat, bendamustine hydrochloride, bexxar (tositumomab and Iodine I 131 tositumomab), BiCNU (carmustine), blenoxane (bleomycin), bleomycin, bortezomib, brentuximab vedotin, carmubris (carmustine), carmustine, chlorambucil, clafen (cyclophosphamide), cyclophosphamide, cytoxan (cyclophosphamide), denileukin diftitox, DepoCyt (liposomal cytarabine), doxorubicin hydrochloride, folex (methotrexate), folotyn (pralatrexate), ibritumomab tiuxetan, ibrutinib, idelalisib, imbruvica (ibtrutinib), intron A (recombinant interferon Alfa-2b), istodax (romidepsin), lenalidomide, leukeran (chlorambucil), linfolizin (chlorambucil), liposomal cytarabine, mechlorethamine hydrochloride, methotrexate, methotrexate LPF (methotrexate), mexate (methotrexate), mexate-AQ (methotrexate), mozobil (perixafor), mustargen (mechlorethamine hydrochloride), nelarabine, neosar (cyclophosphamide), ontak (denifleukin diftitox), perixafor, pralatrexate, prednisone, recombinant interferon Alfa-2b, revlimid (lenalidomide), rituxan (rituximab), rituximab, romidepsin, tositumomab and iodine I 131 tositumomab, treanda (bendamustine hydrochloride), velban (vinblastine sulfate), velcade (bortezomib), velsar (vinblasinte sulfate), vinblastine sulfate, vincasar PFS (vincristine sulfate), vincristine sulfate, vorinostat, zevalin (ibritumomab triuxetan), zolinza (vorinostat), and zydelig (idelalisib).

In one embodiment, the at least one additional agent is a therapeutic agent. In one embodiment, the therapeutic agent is an anti-cancer agent. In one embodiment, the anti-cancer agent is vemurafenib. In one embodiment, an apilimod composition is administered along with vemurafenib in a single dosage form or in separate dosage forms. In one embodiment, the dosage form is an oral dosage form. In another embodiment, the dosage form is suitable for intravenous administration.

In one embodiment, the anti-cancer agent is a drug that is approved for use in treating melanoma. Non-limiting examples of such drugs include aldesleukin, dabrafenib, dacarbazine, DTIC-Dome (darcarbazine), intronA (recombinant interferon Alsfa-2b), ipilimumab, keytruda (pembrolizumab), mekinist (trametinib), nivolumab, peginterferon alfa-2b, PEG-Intron (peginterferon alfa-2b), pembrolizumab, proleukin (aldesleukin), recombinant interferon alfa-2b, sylatron (pegonterferon alfa-2b), tafinlar (dabrafenib), trametinib, vemurafenib, yervoy (ipilimumab), zelboraf (vermurafenib).

In one embodiment, the anti-cancer agent is selected from an inhibitor of EZH2, e.g., EPZ-6438. In one embodiment, the anti-cancer agent is selected from taxol, vincristine, doxorubicin, temsirolimus, carboplatin, ofatumumab, rituximab, and combinations thereof.

In one embodiment, the at least one additional agent is a B cell receptor pathway inhibitor. In some embodiments, the B cell receptor pathway inhibitor is a CD79A inhibitor, a CD79B inhibitor, a CD 19 inhibitor, a Lyn inhibitor, a Syk inhibitor, a PI3K inhibitor, a Blnk inhibitor, a PLCy inhibitor, a PKCP inhibitor, or a combination thereof. In some embodiments, the at least one additional agent is an antibody, B cell receptor signaling inhibitor, a PI3K inhibitor, an IAP inhibitor, an mTOR inhibitor, a radioimmunotherapeutic, a DNA damaging agent, a proteosome inhibitor, a histone deacetylase inhibitor, a protein kinase inhibitor, a hedgehog inhibitor, an Hsp90 inhibitor, a telomerase inhibitor, a Jakl/2 inhibitor, a protease inhibitor, a PKC inhibitor, a PARP inhibitor, or a combination thereof.

In one embodiment, the at least one additional agent is selected from chlorambucil, ifosphamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

In one embodiment, the at least one additional agent is a monoclonal antibody such as, for example, alemtuzumab, bevacizumab, catumaxomab, cetuximab, edrecolomab, gemtuzumab, ofatumumab, panitumumab, rituximab, trastuzumab, eculizumab, efalizumab, muromab-CD3, natalizumab, adalimumab, afelimomab, certolizumab pegol, golimumab, infliximab, basiliximab, canakinumab, daclizumab, mepolizumab, tocilizumab, ustekinumab, ibritumomab tiuxetan, tositumomab, abagovomab, adecatumumab, alemtuzumab, anti-CD30 monoclonal antibody Xmab2513, anti-MET monoclonal antibody MetMab, apolizumab, apomab, arcitumomab, basiliximab, bispecific antibody 2B1, blinatumomab, brentuximab vedotin, capromab pendetide, cixutumumab, claudiximab, conatumumab, dacetuzumab, denosumab, eculizumab, epratuzumab, ertumaxomab, etaracizumab, figitumumab, fresolimumab, galiximab, ganitumab, gemtuzumab ozogamicin, glembatumumab, ibritumomab, inotuzumab ozogamicin, ipilimumab, lexatumumab, lintuzumab, lintuzumab, lucatumumab, mapatumumab, matuzumab, milatuzumab, monoclonal antibody CC49, necitumumab, nimotuzumab, ofatumumab, oregovomab, pertuzumab, ramacurimab, ranibizumab, siplizumab, sonepcizumab, tanezumab, tositumomab, trastuzumab, tremelimumab, tucotuzumab celmoleukin, veltuzumab, visilizumab, volociximab, and zalutumumab.

In the context of combination therapy, administration of the composition may be simultaneous with or sequential to the administration of the one or more additional active agents. In another embodiment, administration of the different components of a combination therapy may be at different frequencies. The one or more additional agents may be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a compound of the present invention.

The one or more additional active agents can be formulated for co-administration with a composition of the invention in a single dosage form, as described in greater detail infra. The one or more additional active agents can be administered separately from the dosage form that comprises the composition of the present invention. When the additional active agent is administered separately, it can be by the same or a different route of administration as the apilimod composition.

Preferably, the administration of a composition as described herein in combination with one or more additional agents provides a synergistic response in the subject being treated. In this context, the term “synergistic” refers to the efficacy of the combination being more effective than the additive effects of either single therapy alone. The synergistic effect of a combination therapy according to the invention can permit the use of lower dosages and/or less frequent administration of at least one agent in the combination compared to its dose and/or frequency outside of the combination. Additional beneficial effects of the combination can be manifested in the avoidance or reduction of adverse or unwanted side effects associated with the use of either therapy in the combination alone (also referred to as monotherapy).

“Combination therapy” also embraces the administration of the compounds of the present invention in further combination with non-drug therapies (e.g., surgery or radiation treatment). Where the combination therapy further comprises a non-drug treatment, the non-drug treatment may be conducted at any suitable time so long as a beneficial effect from the co-action of the combination of the therapeutic compounds and non-drug treatment is achieved. For example, in appropriate cases, the beneficial effect is still achieved when the non-drug treatment is temporally removed from the administration of the therapeutic compounds, perhaps by days or even weeks.

The non-drug treatment can be selected from chemotherapy, radiation therapy, hormonal therapy, anti-estrogen therapy, gene therapy, and surgery. For example, a non-drug therapy is the removal of an ovary (e.g., to reduce the level of estrogen in the body), thoracentesis (e.g., to remove fluid from the chest), paracentesis (e.g., to remove fluid from the abdomen), surgery to remove or shrink angiomyolipomas, lung transplantation (and optionally with an antibiotic to prevent infection due to transplantation), or oxygen therapy (e.g., through a nasal cannula containing two small plastic tubes or prongs that are placed in both nostrils, through a face mask that fits over the nose and mouth, or through a small tube inserted into the windpipe through the front of the neck, also called transtracheal oxygen therapy).

As used herein, the term “selectively” means tending to occur at a higher frequency in one population than in another population. The compared populations can be cell populations. Preferably, an apilimod composition as described herein acts selectively on hyper-proliferating cells or abnormally proliferating cells, compared to normal cells. As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell possesses normally functioning cell cycle checkpoint control mechanisms. Preferably, an apilimod composition acts selectively to modulate one molecular target (e.g., a target kinase) but does not significantly modulate another molecular target (e.g., a non-target kinase). The invention also provides a method for selectively inhibiting the activity of an enzyme, such as a kinase. Preferably, an event occurs selectively in population A relative to population B if it occurs greater than two times more frequently in population A as compared to population B. An event occurs selectively if it occurs greater than five times more frequently in population A. An event occurs selectively if it occurs greater than ten times more frequently in population A; more preferably, greater than fifty times; even more preferably, greater than 100 times; and most preferably, greater than 1000 times more frequently in population A as compared to population B. For example, cell death would be said to occur selectively in diseased or hyper-proliferating cells if it occurred greater than twice as frequently in diseased or hyper-proliferating cells as compared to normal cells.

Methods of Treating mTOR-Related Diseases and Disorders

The present invention also provides methods of treating mTOR-related diseases, disorders, and conditions, in a subject in need thereof by administering to the subject a therapeutically effective amount of a composition of the invention. Such diseases and disorders may include, for example, a cell proliferative disorder in which mTOR is dysregulated, including but not limited to, cancers.

Treating or preventing a cell proliferative disorder according to the methods described herein can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder according to the methods described herein can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder according to the methods described herein can result in a decrease in the size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. The size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder according to the methods described herein can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

### Methods for Treating Charcot-Marie-Tooth Disease

Charcot-Marie-Tooth disease (CMT) is one of the most common inherited neurological disorders, affecting approximately 1 in 2,500 people in the United States. The disease is named for the three physicians who first identified it in 1886—Jean-Martin Charcot and Pierre Marie in Paris, France, and Howard Henry Tooth in Cambridge, England. CMT, also known as hereditary motor and sensory neuropathy (HMSN) or peroneal muscular atrophy, comprises a group of disorders that affect peripheral nerves. Although much research has been undertaken in this field, there are currently no effective treatment options available to patients beyond what is essentially palliative care. Current clinical trials within the United States are investigating substances like coenzyme Q, ascorbic acid and PXT3003, which have shown promise in animal models of neurological disorders.

Alterations in phosphoinositol (PI) signaling and vesicle trafficking have been implicated in CMT disease. Neurons seem to be particularly sensitive to the levels of PI(3,5)P₂, as evidenced by mutations in PI(3,5)P₂-related genes which are implicated in multiple neurological disorders. PI(3,5)P₂ also plays a role in controlling synapse function and/or plasticity. As noted above, PI(3,5)P₂ is generated from PI3P by PIKfyve. An imbalance of PI(3,5)P₂ in Schwann cells has been implicated in causing myelin outfoldings in MTMR2-null nerves. These myelin outfoldings in the nerves consist of redundant loops of myelin around a main myelinated axon and are a hallmark of CMT4B disorders. Genetic and pharmacological inhibition of PIKfyve rescues myelin outfoldings both in vitro and in vivo.

There present invention provides methods for treating CMT by inhibiting PIKfyve. Accordingly, in one aspect, the invention provides a method for treating CMT in a subject in need thereof, the method comprising administering to the subject a therapeutically effective amount of a composition comprising an active metabolite of apilimod, said composition comprising the active metabolite of apilimod in its free base form, or a pharmaceutically acceptable salt, solvate, clathrate, hydrate, polymorph, prodrug, analog or derivative thereof. In one embodiment, the composition comprises the active metabolite of apilimod in its free base form, or a dimesylate salt form. In one embodiment, the CMT is a subtype selected from the group consisting of CMT1, CMT2, CMT3, CMT4, and CMTX. In one embodiment, the CMT is CMT4. In one embodiment, the method further comprises administering at least one additional active agent to the subject. The at least one additional active agent may be a therapeutic agent or a non-therapeutic agent. The at least one additional active agent may be administered in a single dosage form with the composition, or in a separate dosage form from the composition. In one embodiment, the at least one additional active agent is selected from the group consisting of an analgesic agent, a progesterone antagonist, a histone deacetylase inhibitor, a tricyclic antidepressant, anticonvulsant and combinations thereof. In one embodiment, the at least one additional active agent is a therapeutic agent selected from the group consisting of ibuprofen, acetaminophen, naproxen, onapristone, desipramine, doxepin, nortriptyline, amitriptyline, gabapentin, and combinations thereof. In one embodiment, the at least one additional active agent is a non-therapeutic agent selected to ameliorate one or more side effects of the apilimod composition. In one embodiment, the non-therapeutic agent is selected from the group consisting of ondansetron, granisetron, dolasetron and palonosetron. In one embodiment, the non-therapeutic agent is selected from the group consisting of pindolol and risperidone. In one embodiment, the at least one additional active agent is a non-therapeutic agent selected to ameliorate one or more symptoms CMT. In one embodiment, the non-therapeutic agent is selected from the group consisting of physical therapy, stem cell therapy, gene therapy, physiotherapy, and combinations thereof.

In one embodiment, the dosage form of the composition is an oral dosage form. In another embodiment, the dosage form of the composition is suitable for intravenous administration. In one embodiment, where the dosage form is suitable for intravenous administration, administration is by a single injection or by a drip bag.

In one embodiment, the subject is a human CMT patient. In one embodiment, the human CMT patient in need of treatment is one who has been diagnosed with CMT or presents with one or more CMT-related symptoms.

In one embodiment, the method is a method of treating CMT using a combination therapy comprising a composition described herein and an analgesic agent for the treatment of the CMT.

The invention also provides methods of reducing PI(3,5)P₂ levels in neuronal cells, the method comprising delivering to the cells a composition as described herein in an amount effective to selectively inhibit PIKfyve activity in the neuronal cells.

### Methods for Treating Viral Infections

The present invention also provides methods for the treatment and/or prophylaxis of viral infections in a subject, preferably a human subject, in need of such treatment or prevention. In one embodiment, the invention provides a method for treating or preventing a viral infection in a subject in need thereof, the method comprising administering to the subject a composition comprising a therapeutically effective amount of at least one active metabolite of apilimod, as described herein, the therapeutically effective amount being an amount effective to inhibit PIKfyve. In one embodiment, the composition is administered with one or more additional PIKfyve inhibitors selected from the group consisting of apilimod, APY0201, and YM-201636.

In one embodiment, the viral infection is caused by an Ebola virus or a Marburg virus. In one embodiment, the virus is an Ebola virus. In one embodiment, the Ebola virus belongs to a strain selected from the group consisting of the Bundibugyo, Sudan, Tai Forest, and Zaire strains. In one embodiment, the Ebola virus is a Zaire ebola virus.

In one embodiment, the composition is administered orally, for example in the form of a tablet or capsule. In one embodiment, the composition is administered by injection or by addition to sterile infusion fluids for intravenous infusion and is in the form of a suitable sterile aqueous solution or dispersion.

In one embodiment, the therapeutically effective amount of the composition in humans is from about 70 to 1000 mg/day, from about 70 to 500 mg/day, from about 70 to 250 mg/day, from about 70 to 200 mg/day, from about 70 to 150 mg/day, of from about 70 to 100 mg/day.

The present invention also provides methods of treating or preventing a viral infection, or ameliorating one or more symptoms or complications of a viral infection, by administering to a subject, preferably a human subject, a composition comprising a therapeutically effective amount of at least one active metabolite of apilimod, and further comprising administering to the subject at least one additional anti-viral agent, either in the same composition, or in a different composition, for example in a therapeutic regimen as part of a combination therapy for treatment of the viral infection. In one embodiment, the at least one additional anti-viral agent comprises an antibody or a combination of antibodies, preferably human or humanized antibodies, but chimeric (e.g., mouse-human chimeras) antibodies are also acceptable. In one embodiment, the at least one additional anti-viral agent comprises a small interfering RNA (siRNA) or a combination of siRNA molecules. In one embodiment, the siRNA or combination of siRNA molecules targets one or more Ebola virus proteins. In one embodiment, the one or more Ebola virus proteins is selected from the group consisting of the Zaire Ebola L polymerase, Zaire Ebola membrane-associated protein (VP24), and Zaire Ebola polymerase complex protein (VP35). In one embodiment, the siRNA or combination of siRNA molecules targets all three of these proteins.

In the methods described here, composition can be administered by any suitable route and either in the same dosage form or in a different dosage form from the optional additional anti-viral agent or other optional therapeutic agent as described infra. In one embodiment, administration is via an oral, intravenous, or subcutaneous route. In one embodiment, the administration of the composition is once daily, twice daily, or continuous for a period of time, for example one or several days or one or several weeks. Continuous administration may be performed, for example, by using slow release dosage form that is e.g., implanted in the subject, or via continuous infusion, for example using a pump device, which also may be implanted.

In one embodiment, the composition is administered in an amount of 70 to 1000 mg/day. In one embodiment, administration is effective to achieve a plasma concentration of the at least one active metabolite of apilimod in the subject in the range of from 50 to 1000 nM.

In accordance with the methods described herein, the effective amount is, for example, an amount effective to prevent or ameliorate a cytokine storm in the subject, inhibit or reduce the rate of viral replication in the subject, and/or stabilize or reduce the viral load of the subject.

### Pharmaceutical Compositions and Formulations

The present invention provides compositions that are preferably pharmaceutically acceptable compositions suitable for use in a mammal, preferably a human. In this context, the compositions may further comprise at least one pharmaceutically acceptable excipient or carrier, wherein the amount is effective for the treatment of a disease or disorder.

In one embodiment, the composition is combined with at least one additional active agent in a single dosage form. In one embodiment, the composition further comprises an antioxidant.

In one embodiment, the at least one additional active agent is selected from the group consisting of an alkylating agent, an intercalating agent, a tubulin binding agent, a corticosteroid, and combinations thereof. In one embodiment, the at least one additional active agent is a therapeutic agent selected from the group consisting of ibrutinib, rituximab, doxorubicin, prednisolone, vincristine, velcade, and everolimus, and combinations thereof. In one embodiment, the at least one additional active agent is a therapeutic agent selected from cyclophosphamide, hydroxydaunorubicin (also referred to as doxorubicin or Adriamycin™) vincristine (also referred to as Oncovin™), prednisone, prednisolone, and combinations thereof. In one embodiment, the at least one additional active agent is a non-therapeutic agent selected to ameliorate one or more side effects of the apilimod composition. In one embodiment, the non-therapeutic agent is selected from the group consisting of ondansetron, granisetron, dolasetron and palonosetron. In one embodiment, the non-therapeutic agent is selected from the group consisting of pindolol and risperidone.

In one embodiment, the at least one additional active agent is selected from an inhibitor of the mTOR pathway, a PI3K inhibitor, a dual PI3K/mTOR inhibitor, a SRC inhibitor, a VEGF inhibitor, a Janus kinase (JAK) inhibitor, a Raf inhibitor, an Erk inhibitor, a farnesyltransferase inhibitor, a histone deacetylase inhibitor, an anti-mitotic agent, a multi-drug resistance efflux inhibitor, an antibiotic, and a therapeutic antibody. In one embodiment, the at least one additional active agent is selected from a farnesyltransferase inhibitor (e.g., tipifarnib), an anti-mitotic agent (e.g., docetaxel), a histone deacetylase inhibitor (e.g., vorinostat), and a multi-drug resistance efflux inhibitor.

In one embodiment, the mTOR inhibitor is selected from the group consisting of rapamycin (also referred to as sirolimus), everolimus, temsirolimus, ridaforolimus, umirolimus, zotarolimus, AZD8055, INK128, WYE-132, Torin-1, pyrazolopyrimidine analogs PP242, PP30, PP487, PP121, KU0063794, KU-BMCL-200908069-1, Wyeth-BMCL-200910075-9b, INK-128, XL388, AZD8055, P2281, and P529. See, e.g., Liu et al. Drug Disc. Today Ther. Strateg., 6(2): 47-55 (2009).

In one embodiment, the mTOR inhibitor is trans-4-[4-amino-5-(7-methoxy-1H-indol-2-yl)imidazo[5,1-f][1,2,4]triazin-7-yl]cyclohexane carboxylic acid (also known as OSI-027), and any salts, solvates, hydrates, and other physical forms, crystalline or amorphous, thereof. See US 2007/0112005. OSI-027 can be prepared according to US 2007/0112005, incorporated herein by reference. In one embodiment, the mTOR inhibitor is OXA-01. See e.g., WO 2013152342 A1.

In one embodiment, the PI3K inhibitor is selected from the group consisting of GS-1101 (Idelalisib), GDC0941 (Pictilisib), LY294002, BKM120 (Buparlisib), PI-103, TGX-221, IC-87114, XL 147, ZSTK474, BYL719, AS-605240, PIK-75, 3-methyladenine, A66, PIK-93, PIK-90, AZD6482, IPI-145 (Duvelisib), TG100-115, AS-252424, PIK294, AS-604850, GSK2636771, BAY 80-6946 (Copanlisib), CH5132799, CAY10505, PIK-293, TG100713, CZC24832 and HS-173.

In one embodiment, the dual PI3K/mTOR inhibitor is selected from the group consisting of, GDC-094, WAY-001, WYE-354, WAY-600, WYE-687, Wyeth-BMCL-200910075-16b, Wyeth-BMCL-200910096-27, KU0063794 and KUBMCL-200908069-5, NVP-BEZ235, XL-765, PF-04691502, GDC-0980 (Apitolisib), GSK1059615, PF-05212384, BGT226, PKI-402, VS-558 and GSK2126458. See, e.g., Liu et al. Drug Disc. Today Ther. Strateg., 6(2): 47-55 (2009), incorporated herein by reference.

In one embodiment, the mTOR pathway inhibitor is a polypeptide (e.g., an antibody or fragment thereof) or a nucleic acid (e.g., a double-stranded small interfering RNA, a short hairpin RNA, a micro-RNA, an antisense oligonucleotide, a locked nucleic acid, or an aptamer) that binds to and inhibits the expression level or activity or a protein (or nucleic acid encoding the protein) in the mTOR pathway. For example, the polypeptide or nucleic acid inhibits mTOR Complex 1 (mTORC1), regulatory-associated protein of mTOR (Raptor), mammalian lethal with SEC13 protein 8 (MLST8), proline-rich Akt substrate of 40 kDa (PRAS40), DEP domain-containing mTOR-interacting protein (DEPTOR), mTOR Complex 2 (mTORC2), rapamycin-insensitive companion of mTOR (RICTOR), G protein beta subunit-like (GβL), mammalian stress-activated protein kinase interacting protein 1 (mSIN1), paxillin, RhoA, Ras-related C3 botulinum toxin substrate 1 (Rac1), Cell division control protein 42 homolog (Cdc42), protein kinase C α (PKCα), the serine/threonine protein kinase Akt, phosphoinositide 3-kinase (PI3K), p70S6K, Ras, and/or eukaryotic translation initiation factor 4E (eIF4E)-binding proteins (4EBPs), or the nucleic acid encoding one of these proteins.

In one embodiment, the SRC inhibitor is selected from the group consisting of bosutinib, saracatinib, dasatinib, ponatinib, KX2-391, XL-228, TG100435/TG100855, and DCC2036. See, e.g., Puls et al. Oncologist. 2011 May; 16(5): 566-578. In one embodiment, the SRC inhibitor is a polypeptide (e.g., an antibody or fragment thereof) or nucleic acid (e.g., a double-stranded small interfering RNA, a short hairpin RNA, a micro-RNA, an antisense oligonucleotide, a locked nucleic acid, or an aptamer) that binds to and inhibits the expression level or activity of the SRC protein or a nucleic acid encoding the SRC protein.

In one embodiment, the VEGF inhibitor is selected from bevacizumab, sunitinib, pazopanib, axitinib, sorafenib, regorafenib, lenvatinib, and motesanib. In one embodiment, the VEGF inhibitor is a polypeptide (e.g., an antibody or fragment thereof) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of a VEGF protein, a VEGF receptor protein, or a nucleic acid encoding one of these proteins. For example, the VEGF inhibitor is a soluble VEGF receptor (e.g., a soluble VEGF-C/D receptor (sVEGFR-3)).

In one embodiment, the JAK inhibitor is selected from facitinib, ruxolitinib, baricitinib, CYT387 (CAS number 1056634-68-4), lestaurtinib, pacritinib, and TG101348 (CAS number 936091-26-8). In one embodiment, the JAK inhibitor is a polypeptide (e.g., an antibody or fragment thereof) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of a JAK (e.g., JAK1, JAK2, JAK3, or TYK2) or a nucleic acid encoding the JAK protein.

In one embodiment, the Raf inhibitor is selected from PLX4032 (vemurafenib), sorafenib, PLX-4720, GSK2118436 (dabrafenib), GDC-0879, RAF265, AZ 628, NVP-BHG712, SB90885, ZM 336372, GW5074, TAK-632, CEP-32496 and LGX818 (Encorafenib). In one embodiment, the Raf inhibitor is a polypeptide (e.g., an antibody or fragment thereof) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of a Raf (e.g., A-Raf, B-Raf, C-Raf) or a nucleic acid encoding the Raf protein. In one embodiment, the MEK inhibitor is selected from AZD6244 (Selumetinib), PD0325901, GSK1120212 (Trametinib), U0126-EtOH, PD184352, RDEA119 (Rafametinib), PD98059, BIX 02189, MEK162 (Binimetinib), AS-703026 (Pimasertib), SL-327, BIX02188, AZD8330, TAK-733 and PD318088. In one embodiment, the MEK inhibitor is a polypeptide (e.g., an antibody or fragment thereof) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of a MEK (e.g., MEK-1, MEK-2) or a nucleic acid encoding the MEK protein.

In one embodiment, the Akt inhibitor is selected from MK-2206, KRX-0401 (perifosine), GSK690693, GDC-0068 (Ipatasertib), AZD5363, CCT128930, A-674563, PHT-427. In one embodiment, the Akt inhibitor is a polypeptide (e.g., an antibody or fragment thereof) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of a Akt (e.g., Akt-1, Akt-2, Akt-3) or a nucleic acid encoding the Akt protein.

In one embodiment, the farnesyltransferase inhibitor is selected from LB42708 or tipifarnib. In one embodiment, the farnesyltransferase inhibitor is a polypeptide (e.g., an antibody or fragment thereof) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of farnesyltransferase or a nucleic acid encoding the farnesyltransferase protein. In one embodiment, the hi stone modulating inhibitor is selected from anacardic acid, C646, MG149 (histone acetyltransferase), GSK J4 Hcl (histone demethylase), GSK343 (active against EZH2), BIX 01294 (histone methyltransferase), MK0683 (Vorinostat), MS275 (Entinostat), LBH589 (Panobinostat), Trichostatin A, MGCD0103 (Mocetinostat), Tasquinimod, TMP269, Nexturastat A, RG2833, PDX101 (Belinostat).

In one embodiment, the anti-mitotic agent is selected from Griseofulvin, vinorelbine tartrate, paclitaxel, docetaxel, vincristine, vinblastine, Epothilone A, Epothilone B, ABT-751, CYT997 (Lexibulin), vinflunine tartrate, Fosbretabulin, GSK461364, ON-01910 (Rigosertib), Ro3280, BI2536, NMS-P937, BI 6727 (Volasertib), HMN-214 and MLN0905.

In one embodiment, the polyether antibiotic is selected from sodium monensin, nigericin, valinomycin, salinomycin.

A “pharmaceutical composition” is a formulation containing the compounds described herein in a pharmaceutically acceptable form suitable for administration to a subject. As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof.

A pharmaceutical composition can be provided in bulk or in dosage unit form. It is especially advantageous to formulate pharmaceutical compositions in dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved. A dosage unit form can be an ampoule, a vial, a suppository, a dragee, a tablet, a capsule, an IV bag, or a single pump on an aerosol inhaler.

In therapeutic applications, the dosages vary depending on the agent, the age, weight, and clinical condition of the recipient patient, and the experience and judgment of the clinician or practitioner administering the therapy, among other factors affecting the selected dosage. Generally, the dose should be a therapeutically effective amount. Dosages can be provided in mg/kg/day units of measurement (which dose may be adjusted for the patient's weight in kg, body surface area in m², and age in years). An effective amount of a pharmaceutical composition is that which provides an objectively identifiable improvement as noted by the clinician or other qualified observer. For example, alleviating a symptom of a disorder, disease or condition. As used herein, the term “dosage effective manner” refers to amount of a pharmaceutical composition to produce the desired biological effect in a subject or cell.

For example, the dosage unit form can comprise 1 nanogram to 2 milligrams, or 0.1 milligrams to 2 grams; or from 10 milligrams to 1 gram, or from 50 milligrams to 500 milligrams or from 1 microgram to 20 milligrams; or from 1 microgram to 10 milligrams; or from 0.1 milligrams to 2 milligrams.

The pharmaceutical compositions can take any suitable form (e.g, liquids, aerosols, solutions, inhalants, mists, sprays; or solids, powders, ointments, pastes, creams, lotions, gels, patches and the like) for administration by any desired route (e.g, pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the invention may be in the form of an aqueous solution or powder for aerosol administration by inhalation or insufflation (either through the mouth or the nose), in the form of a tablet or capsule for oral administration; in the form of a sterile aqueous solution or dispersion suitable for administration by either direct injection or by addition to sterile infusion fluids for intravenous infusion; or in the form of a lotion, cream, foam, patch, suspension, solution, or suppository for transdermal or transmucosal administration.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present invention with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. In the case of tablets for oral use, carriers which are commonly used include lactose and corn starch. Lubricating agents, such as magnesium stearate, can also be added. For oral administration in a capsule form, useful diluents include lactose and dried corn starch. When aqueous suspensions and/or emulsions are administered orally, the compound of the present invention may be suspended or dissolved in an oily phase is combined with emulsifying and/or suspending agents. If desired, certain sweetening and/or flavoring and/or coloring agents may be added.

A pharmaceutical composition can be in the form of a tablet. The tablet can comprise a unit dosage of a compound of the present invention together with an inert diluent or carrier such as a sugar or sugar alcohol, for example lactose, sucrose, sorbitol or mannitol. The tablet can further comprise a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. The tablet can further comprise binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures.

The tablet can be a coated tablet. The coating can be a protective film coating (e.g. a wax or varnish) or a coating designed to control the release of the active agent, for example a delayed release (release of the active after a predetermined lag time following ingestion) or release at a particular location in the gastrointestinal tract. The latter can be achieved, for example, using enteric film coatings such as those sold under the brand name Eudragit®.

Tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine.

A pharmaceutical composition can be in the form of a hard or soft gelatin capsule. In accordance with this formulation, the compound of the present invention may be in a solid, semi-solid, or liquid form.

A pharmaceutical composition can be in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

A pharmaceutical composition can be in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions of the compound of the present invention as a free base or pharmacologically acceptable salt can be prepared in water suitably mixed with a surfactant. Examples of suitable surfactants are given below. Dispersions can also be prepared, for example, in glycerol, liquid polyethylene glycols and mixtures of the same in oils.

The pharmaceutical compositions for use in the methods of the present invention can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants, the HLB values can differ by as much as about 8 HLB units, depending upon the empirical method chosen to determine the HLB value.

Among the surfactants for use in the compositions of the invention are polyethylene glycol (PEG)-fatty acids and PEG-fatty acid mono and diesters, PEG glycerol esters, alcohol-oil transesterification products, polyglyceryl fatty acids, propylene glycol fatty acid esters, sterol and sterol derivatives, polyethylene glycol sorbitan fatty acid esters, polyethylene glycol alkyl ethers, sugar and its derivatives, polyethylene glycol alkyl phenols, polyoxyethylene-polyoxypropylene (POE-POP) block copolymers, sorbitan fatty acid esters, ionic surfactants, fat-soluble vitamins and their salts, water-soluble vitamins and their amphiphilic derivatives, amino acids and their salts, and organic acids and their esters and anhydrides.

The present invention also provides packaging and kits comprising pharmaceutical compositions for use in the methods of the present invention. The kit can comprise one or more containers selected from the group consisting of a bottle, a vial, an ampoule, a blister pack, and a syringe. The kit can further include one or more of instructions for use in treating and/or preventing a disease, condition or disorder of the present invention, one or more syringes, one or more applicators, or a sterile solution suitable for reconstituting a pharmaceutical composition of the present invention.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

### EXAMPLES

### Example 1: Apilimod is a Highly Selective Inhibitor of TSC2 Null Cell Proliferation

Apilimod was identified in a high throughput cell viability screen using TSC2−/− mouse embryonic fibroblasts (MEF-EV) cells. TSC2 null cells have constitutively active mTOR. Briefly, MEF cells derived from TSC2−/− knockout mouse embryos (Onda et al., J. Clin. Invest. 104(6):687-95, 1999) were infected with a retrovirus vector encoding the hygromycin antibiotic resistance gene (MEF-EV) or the same retrovirus vector also encoding TSC2 (MEF-TSC2). The MEF-EV and MEF-TSC2 line were then established by hygromycin selection.

Cells were expanded in DMEM containing 10% FBS (Omega Scientific) and 2 mM L-Glutamine. Frozen stocks of cells were prepared for direct use in the HTS assay. Cells were harvested, pelleted and then resuspended in 95% FBS & 5% DMSO at a concentration 1×10⁷ cells/ml. One ml aliquots were rate frozen to −80 at a rate of 1 degree per minute. These stocks were then transferred to vapor phase liquid nitrogen for long term storage.

For screening, vials were thawed at 37° C. with continuous agitation until just thawed then re-suspended in room temperature assay media and centrifuged at 1,000 rpm for 5 minutes. The resulting pellet was re-suspended in appropriate volume and counted using an automated cell counter and diluted accordingly to a final count of 40,000 cells/ml.

Test compounds (5 μl stock solution, 6× desired final well concentration) were dispensed to 384-well assay plates (Corning 3712) using a Biomek FX liquid handler. MEF-EV cells (1000 cells per well in 25 μL of media) were added to these pre-formatted plates using a Thermo Wellmate, non-contact dispensing system with a standard bore cassette head. Plates were incubated for 72 h at 37° C. under an atmosphere of 5% CO₂ in a humidified incubator.

Cell viability was determined with CellTiter-Glo® luminescence assay (Promega) as per the manufacturer's instructions. Viability was expressed as a percentage of untreated control cells. As an example, for apilimod, MEF-EV cell viability (Mean+/−StDev, n=3) was 2.16+/−0.36% @ 0.5 μM and 1.94+/−0.07% @ 5 μM.

The activity of apilimod on TSC2 deficient cells was further demonstrated by performing 10 point dose response on the MEF-EV and MEF-TSC2 lines described above as well as three additional pairs of isogenic lines: (1) (TSC2−/−, p53−/−) and (TSC2+/−, p53−/−) MEF lines were established from (TSC2−/−, p53−/−) or (TSC2+/−, p53−/−) embryos according to standard methods. See e.g., Zhang et al. J. Clin. Invest. 112, 1223-33, 2003. (2) ELT3-EV and ELT3-TSC2 lines were established from the ELT3 rat tumor cell line. The ELT3 line is an established rat tumor model for LAM/TSC. See e.g., Howe et al., Am. J. Path. 146, 1568-79, 1995. These cells harbor an inactivating mutation in TSC2, which leads to constitutive activation of the mTOR pathway. To develop an isogenic pair of cells ELT3 cells were infected with a retrovirus vector encoding the hygromycin antibiotic resistance gene (ELT3-EV) or the same retrovirus vector also encoding TSC2 (ELT3-TSC2). The ELT3-EV and ELT3-TSC2 line were then established by hygromycin selection. (3) TRI-AML102 and AML103 lines were established from a TSC2 null primary human AML sample provided by Dr. Elizabeth Henske (Fox Chase Cancer Center, Philadelphia, Pa.). The cells were infected with amphotropic retrovirus LXSN16E6E7 that encodes the HPV16 E6 and E7 open reading frames and neomycin resistance cassette. Cells were expanded and neomycin-selected. Individual clones were isolated and frozen down. The coding sequence for the human Telomerase gene (hTERT) with hygromycin resistance cassette (pLXSN hTERT-hyg plasmid) was stably expressed into a TSC2^{-/-} confirmed E6E7 AML clone using Fugene6 transfection reagent (Roche Applied Science, Indianapolis, Ind.). TRI-AML102 was generated by stable incorporation of a control zeomycin selection plasmid (pcDNA3.1-zeo), while TRI-AML103 expresses the human TSC2 cDNA pcDNA3.1-zeo plasmid. As a result of these engineering processes, both TRI102 and TRI103 are neomycin, hygromycin, and zeomycin resistant lines.

For 10-point dose response, 750 MEF, 2000 ELT3, or 2000 AML cells in 100 μL of growth media (DMEM (CellGro 10-017-CV) FBS 10% (Sigma Aldrich F2442-500 mL, Lot 12D370) Penicillin/Streptomycin (100×) (CellGro Ref 30-002) were plated per well of a 96 well plate. 24 hours after plating cells, the media was removed and apilimod dilutions (1-500 nM, 2-fold dilutions) in 100 μL of growth media were added (0.1% final DMSO concentration). 72 hours after compound addition, relative cell viability was determined by CellTiter-Glo® luminescence assay (Promega) and expressed as a percentage relative to vehicle (DMSO) treated control cells. IC₅₀ values were then calculated from the dose response curves using XLFIT (IDBS).

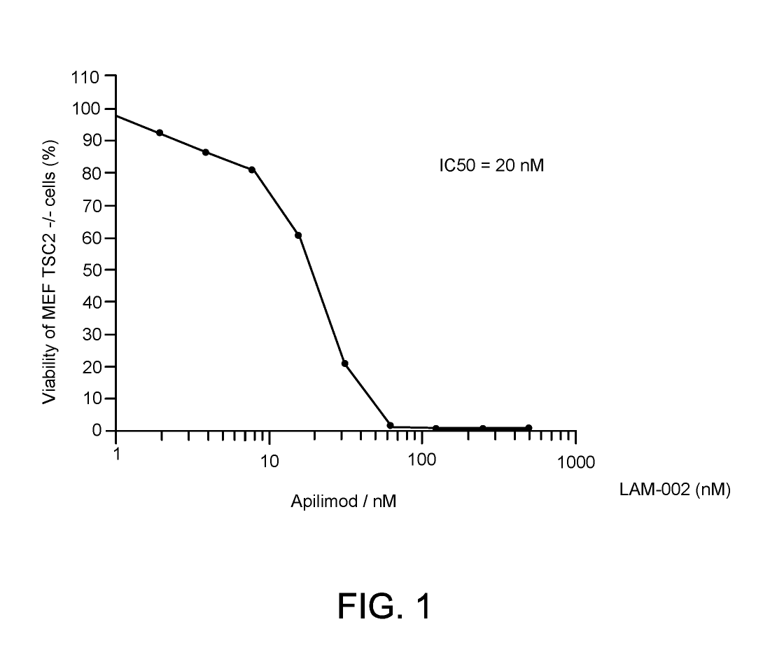

The TSC2 deficient cells were highly sensitive to apilimod (IC₅₀=20 nM, FIG. 1). TSC2−/− p53−/− MEFs demonstrated increased sensitivity to apilimod compared to the TSC2+/−p53−/− MEFs as indicated by a selectivity ratio above 1 (2.45).

TABLE 2
IC₅₀ (viability) of apilimod in various cell types

| | | MEF | AML | |
|---|---|---|---|---|
| | MEF | TSC2 −/− | TSC2 +/− | |
| Cell type: | TSC2 −/− | p53 −/− | p53 −/− | ELT3 |
| IC50 TSC2 −/− | 19.70 | 28.80 | 117.00 | 13.70 |
| IC50 TSC2 rescue | 20.10 | 70.70 | 132.00 | 16.05 |
| Selectivity Ratio | 1.02 | 2.45 | 1.13 | 1.17 |

IC50s (nM) Calculated from 10-Point Dose Response on TSC2−/− Deficient and Rescue Lines.
IC50s are calculated from the average of two experiments. The selectivity ratio is calculated by dividing the IC50 of the TSC2 rescue line by the TSC2−/− line.

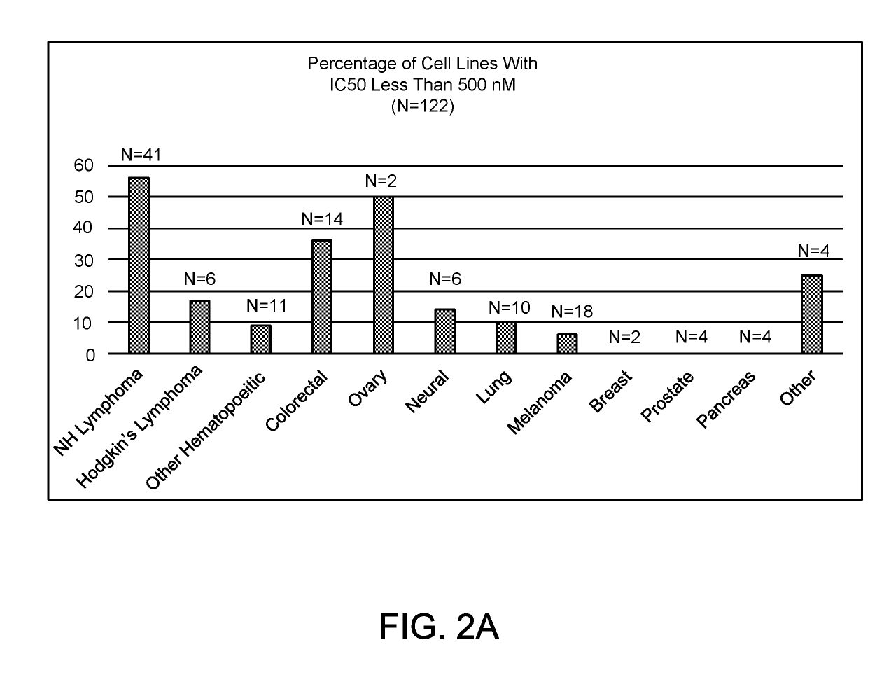

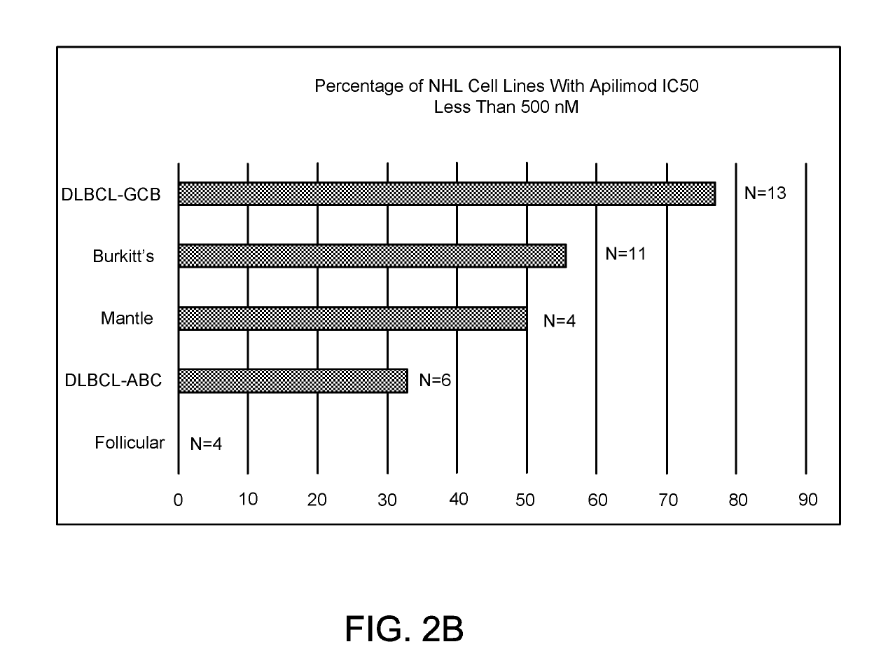

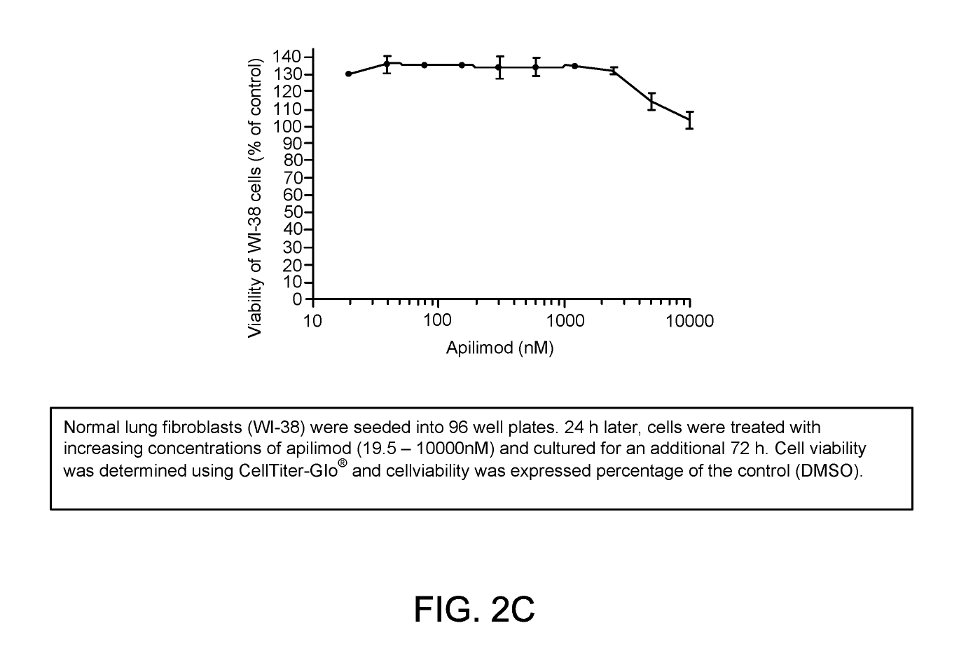

Furthermore, higher concentrations of apilimod had higher potency on the TSC2−/− MEF-EV cells compared to the TSC2 rescue MEF-TSC2 cells. This data, coupled to the fact that apilimod is not cytotoxic on peripheral blood mononuclear cells (Wada et al., Blood 109, 1156-64, 2007), nor on a variety of other cancer lines including U937, HELA, Jurkat, and THP-1 (PCT Publication No. WO 2006/128129), nor on normal lung fibroblasts, suggests that there will be a high therapeutic index for treating TSC2−/− cancer cells with apilimod (FIG. 2A-2C).

### Example 2: Apilimod is a Highly Selective Cytotoxic Agent in Cancer Cells

The cytotoxic activity of apilimod was evaluated using a standard cell viability assay such as CellTiterGlo™ according to the manufacturer's instructions. 122 human cancer cell lines were evaluated for sensitivity to apilimod. A cell line was called as apilimod sensitive if the IC₅₀ was less than 500 nM. 35 cell lines were identified as sensitive to apilimod-induced cytotoxicity. Apilimod was also highly selective for cancer cells compared to normal cells, which had IC₅₀'s ranging from 20-200 fold higher than the cancer cells (FIG. 2A-2C).

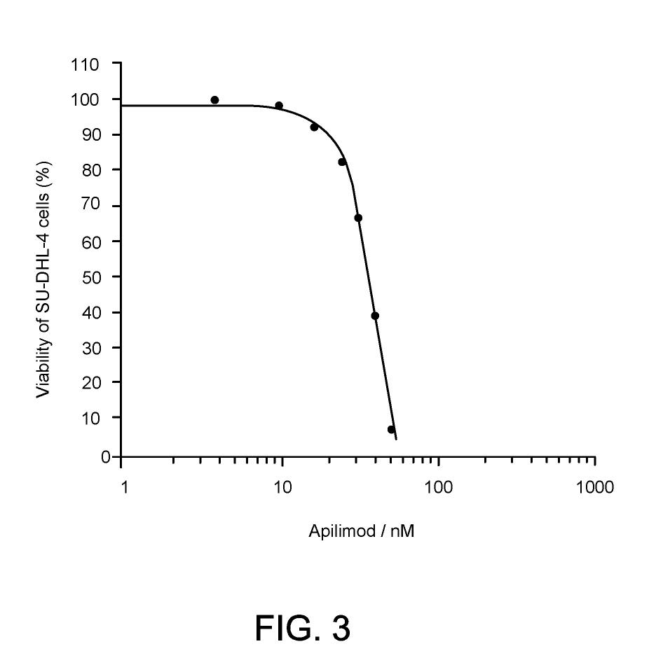

FIG. 2A shows that the apilimod-sensitive cells included cells derived from several different cancers including non-Hodgkin's lymphoma, Hodgkin's lymphoma, colorectal cancer, and lung cancer. The most sensitive of those tested were non-Hodgkin's Lymphoma (NHL) cell lines. Just over 50% of the NHL cell lines tested were sensitive to apilimod. NHL represents a diverse group of hematopoietic malignancies that vary in severity, with subtypes ranging from slow growing to aggressive. Subtypes of NHL include diffuse large B cell lymphoma (DLBCL), Burkitt's lymphoma, mantle lymphoma, and follicular B cell lymphoma. DLBCL is divided into two subtypes, GCB and ABC, based on gene expression and cell of origin. The GCB are germinal center B cell type, arising from normal germinal center B cells, and the ABC are activated B cell type, arising from post-germinal center B cells in the process of differentiating into plasma cells. In the present study, we found that certain subtypes of NHL were extremely sensitive to apilimod, with IC₅₀ values of less than 100 nM (compared to the cutoff for sensitive/insensitive in the screen, which was 500 nM). These included a human Burkitt's lymphoma (ST486), a human mantle cell lymphoma (JeKo-1) and a human DLBCL (SUDHL-4, IC₅₀=50 nM). See FIG. 3. These results indicate that apilimod may be effective against many NHL cancers, including the more aggressive subtypes that are often refractory to standard treatments.

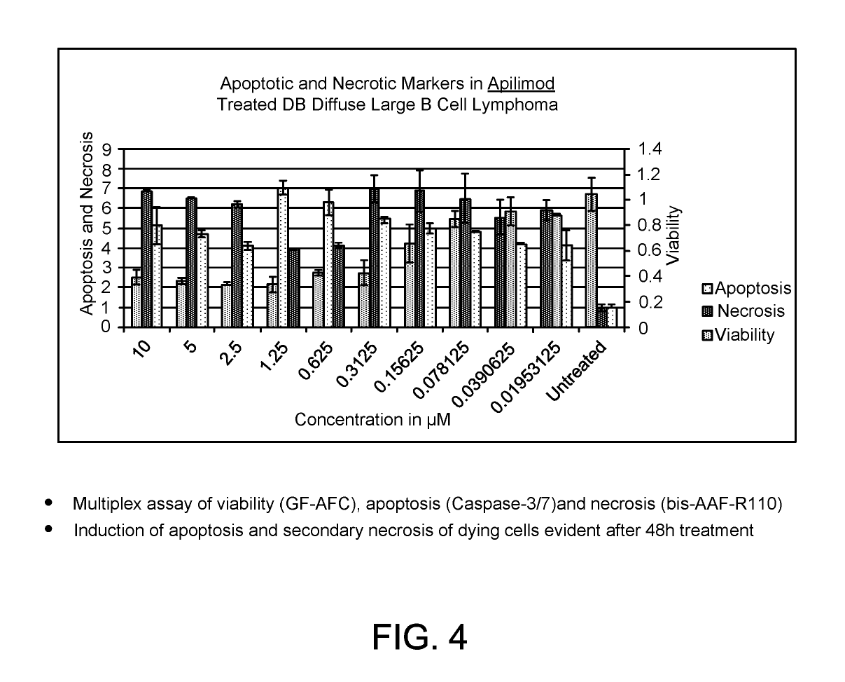

As detailed in Examples 6 and 7, infra, we investigated the biological mechanisms underlying apilimod's selective cytotoxicity against cancers cells and found that it is due to an inhibition of intracellular trafficking and a corresponding increase in apoptosis in those cells. See FIG. 4.

### Example 3: Apilimod Synergizes with Components of CHOP

As discussed above, NHL cells demonstrated particular sensitivity to apilimod in our cancer cell line screen. DLBCL is the most common type of NHL, accounting for 30-40% of lymphomas in Western countries. DLBCL is an aggressive neoplasm of mature B cells. Approximately 40% of all DLBCL patients relapse after first line treatment. Many refractory DLBCL-GCB cancers exhibit single and double translocations of MYC and BCL2. Patients with these genetic variants tend to have a poorer prognosis due at least in part to overexpression of MYC and BCL2. Notably, apilimod was effective even in DLBCL-GCB cell lines exhibiting these translocations (Table 3), supporting a role for apilimod in the treatment of even aggressive subtypes of NHL, either alone, as monotherapy, or in combination with standard treatments.

TABLE 3
Bcl-2 and c-myc translocation status for B Cell Lymphoma
Lines and their sensitivity to apilimod. ND = No Data

| | B Cell Lymphoma | | IC₅₀ | | |
|---|---|---|---|---|---|
| Number | Model | Cell Line | (nM) | Bcl-2 | C-myc |
| 7 | Human DLBCL-GCB | SUDHL-4 | 25 | Yes | Yes |
| 8 | Human DLBCL-GCB | SUDHL-6 | 80 | Yes | No |
| 9 | Human DLBCL-GCB | DB | 150 | No | No |
| 10 | Human DLBCL-GCB | Toledo | 270 | ND | ND |
| 11 | Human DLBCL-GCB | SUDHL-10 | 20 | Yes | Yes |
| 12 | Human DLBCL-GCB | WSU-DLCL2 | 160 | Yes | No |
| 13 | Human DLBCL-GCB | OCI-Ly19 | 380 | Yes | No |
| 20 | Human DLBCL-GCB | HT | 642 | ND | ND |
| 21 | Human DLBCL-GCB | Pfeiffer | 2,620 | ND | ND |

To further evaluate the effectiveness of apilimod against aggressive NHL tumors, the ability of apilimod to act synergistically with any of a number of chemotherapeutic agents that comprise the standard first line treatment for many such cancers was tested. These included, for example, cyclophosphamide, doxorubicin, vincristine and prednisone (referred to as the “CHOP” chemotherapy regimen), and rituximab, which is sometimes combined with CHOP (R-CHOP), as well as the chemotherapeutic agents velcade, which is indicated for relapsed mantle cell lymphoma, and everolimus, an inhibitor of mTOR.

For synergy studies the following DLBCL-GCB cell lines were used: SUDHL-4, SUDHL-5, and SUDHL-6. Cells were seeded in 96 well plates at their optimum density. Cells were treated with apilimod alone (7.8-1000 nM), cyclophosphamide (mafosfamide; 78-10000 nM), doxorubicin (3.13-400 nM), vincristine (0.08-10 nM), prednisone (19.5-2500 nM), velcade (0.16-20 nM), or everolimus (0.23-500 nM), either alone or in a combination with apilimod. In each case, the dilutions were 2-fold with a total of 8 dilutions over the drug concentration range.

Cells were treated for 72 h before proliferation was assessed using CellTiterGlo® (Promega). For calculation of synergy, CalcuSyn (version 2.11, Biosoft) was used to determine the combination index (CI) as defined by Chou et al., Adv. Enzyme. Regul. (1984) 22:27-55. Thus, drug combinations producing CI values >1 were defined as antagonistic, CI=1 as additive, and CI<1 as synergistic.

As shown in Table 4, apilimod demonstrated synergistic activity with 5 of 6 agents tested (doxorubicin, prednisolone, vincristine, velcade, and everolimus) in the SUDHL-6 cell line and was synergistic with vincristine in all three cell lines. In addition, apilimod was synergistic with prednisolone, velcade, and everolimus in at least two of the three cell lines tested. These results demonstrate that combination therapy with apilimod represents a promising new approach for addressing the unmet medical need for treatments that benefit patients who relapse after or who are refractory to standard chemotherapy regimens.

TABLE 4

| Combination Treatment | | | SU-DHL-5 | SU-DHL-4 | SU-DHL-6 |
|---|---|---|---|---|---|
| Standard | Mafosfamide | Apilimod | Synergistic | Synergistic | Synergistic |
| of care | 625-2500 nM | 62.5-250 nM | | | |
| | Doxorubicin | Apilimod | Synergistic | Synergistic | Synergistic |
| | 50-400 nM | 125-500 nM | | | |
| | Prednisolone | Apilimod | Synergistic | Synergistic | Synergistic |
| | 62-1667 nM | 62.5-500 nM | | | |
| | Vincristine | Apilimod | Synergistic | Synergistic | Synergistic |
| | 0.6-10 nM | 62.5-1000 nM | | | |
| Other | Velcade | Apilimod | Synergistic | Synergistic | Synergistic |
| therapies | 1.3-5 nM | 62.5-250 nM | | | |
| | Everolimus | Apilimod | ND | ND | Synergistic |
| | 18.5-55.6 nM | 62.5-250 nM | | | |

Summary of drug combination effects of apilimod and individual components of CHOP (mafosfamide used instead of cyclophosphamide), Velcade or Everolimus in DLBCL-GCB cell lines. Combination index (CI) was used to determine combination effects, where CI>1 is antagonistic, CI=1 is additive and CI<1 is synergistic. The range of concentrations of apilimod in combination with either CHOP components, Velcade or Everolimus to produce the described effect is shown (italics).

### Example 4: Synergistic Activity Between Apilimod and Ibrutinib

Studies in SUDHL-4 cells were also undertaken to screen for other drugs that could act synergistically with apilimod. A manually curated library of 93 drugs including both FDA approved and unapproved drugs was used in the screen. Cells were grown in the presence of drug, with or without apilimod (at IC₂₀=10 nM), with each drug of the library being tested in a 10 point-concentration response curve (1.5-30,000 nM; 3-fold dilutions). SUDHL-4 cells were grown in RPMI Medium 1640 containing (Sigma Aldrich F2442-500 mL, Lot 12D370) Penicillin/Streptomycin (100×) (CellGro Ref 30-002). Cells were seeded into 96 well plates at a density of 19,000 cells per well, in a final volume of 50 μL. 50 μL of the 10 point drug dilution series (at 2×) was added to the cells to give the final concentrations stated above. Plates were incubated at 37° C. under an atmosphere of 5% CO₂ in a humidified incubator. 72 hours after compound addition relative cell viability was determined by CellTiter-Glo® luminescence assay (Promega) as per the manufacturer's instructions, and values were expressed as a percentage relative to vehicle (DMSO) treated control cells (set to 100%).

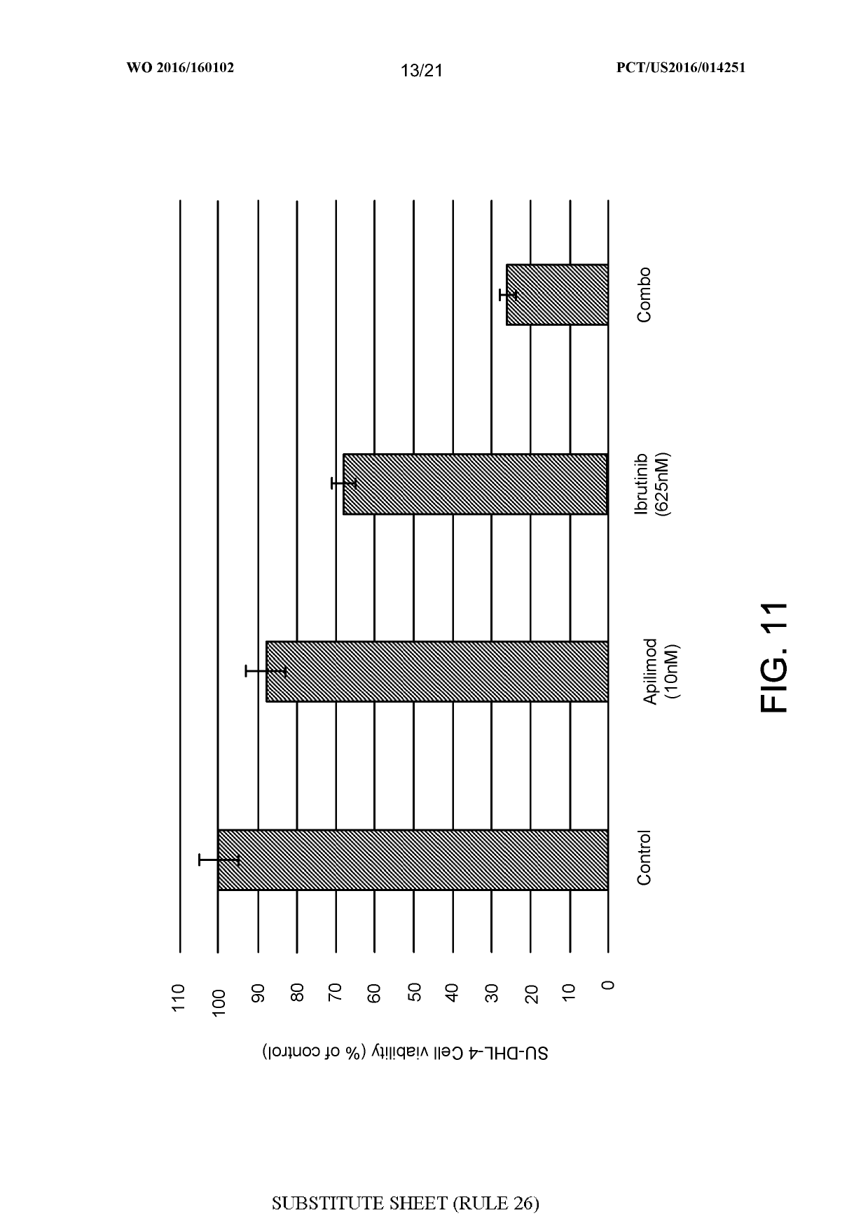

The viability of cells treated with individual compound in the drug library was compared to the viability of cells treated with each library drug+apilimod (IC₂₀) and significant combinations were identified. Ibrutinib was identified as significantly reducing SUDHL-4 cell viability in the presence of apilimod compared with either ibrutinib or apilimod alone. See FIG. 11. Ibrutinib is an FDA-approved drug targeting B-cell malignancies and indicated for monotherapy in treating mantle cell lymphoma and chronic lymphocytic leukemia. It is also known as PCI-32765 and marketed under the trade name Imbruvica™. Ibrutinib is a selective and covalent inhibitor of the enzyme Bruton's tyrosine kinase (BTK). BTK is a key mediator of at least three critical B-cell pro-survival mechanisms occurring in parallel-regulation of apoptosis, cell adhesion and cell migration and homing. The synergistic activity of apilimod with ibrutinib further indicate that apilimod is a promising agent for use in combination therapy with other chemotherapy agents, especially those targeted against B-cell lymphomas.

### Example 5: Anti-Tumor Activity of Apilimod in Combination with Ibrutinib on DLBCL Tumors In Vivo

The ability of apilimod to inhibit tumor growth in vivo, either alone or in combination with ibrutinib was tested next. As described below, apilimod alone significantly reduced tumor growth and the combination of apilimod and ibrutinib provided greater growth inhibition than either agent alone.

The study objective was to evaluate pre-clinically the in vivo therapeutic efficacy of apilimod in the treatment of a subcutaneous SUDHL-6 human DLBCL cancer xenograft model alone, and in combination with ibrutinib.

In the first arm of the study, apilimod was tested alone. The SUDHL-6 cell line was maintained in RPMI-1640 medium supplemented with 10% fetal bovine serum and L-glutamine (2 mM) at 37° C. in an atmosphere of 5% CO₂. The tumor cells were sub-cultured twice weekly and harvested during exponential growth for tumor inoculation. NOD-SCID mice were γ-irradiated 24 hrs before inoculation. Each mouse was inoculated subcutaneously in the right flank with SU-DHL-6 tumor cells (5×10⁶) in 0.1 ml of PBS with Matrigel (1:1). The tumors were then grown to a mean size of approximately 80-120 mm³ and the mice were then split into 5 groups and treated as detailed in the Table 5.

TABLE 5
Xenograft Model of DLBCL tumors

| | | | | | Number |
|---|---|---|---|---|---|
| | | | | Administration | of |
| Group | Treatment | Dose | Dosing schedule | route | mice |
| 1 | Vehicle(Saline) | — | QD × 5 −2 days off- | i.v. | 6 |
| | | | QD × 5 | | |
| 2 | Apilimod | 67.5 mg/kg | QD × 5 −2 days off- | i.v. | 6 |
| | Dimesylate | (47 mg/kg | QD × 5 | | |
| | | Free | | | |
| | | Base) | | | |
| 3 | 0.5% | — | BID × 5 −2 days off- | p.o. | 6 |
| | Methylcellulose | | BID × 5 | | |
| 4 | Apilimod Free | 75 mg/kg | BID × 5 −2 days off- | p.o. | 6 |
| | Base | | BID × 5 | | |
| 5 | Apilimod Free | 150 mg/kg | QD × 5 −2 days off- | p.o. | 6 |
| | Base | | BID × 5 | | |

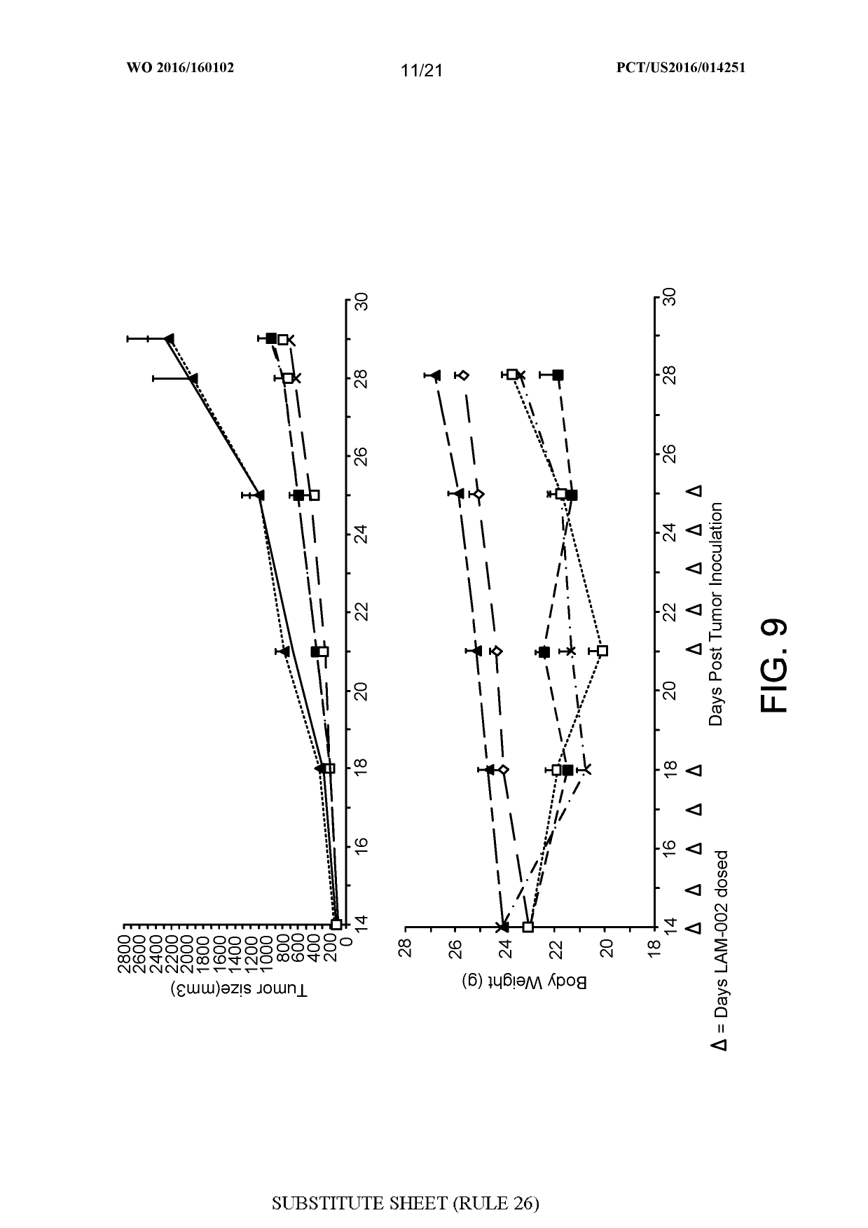

Tumor size was measured twice a week in two dimensions using a caliper, and the volume is expressed in mm³ using the formula: V=0.5 a×b² where a and b are the long and short diameters of the tumor, respectively. The mice were monitored for 29 days and significant growth inhibition was observed in all apilimod treatment arms. Intravenous administration reduced tumor size by 58% (47 mg/kg) and oral dosing reduced growth by 68% (150 mg/kg split dose) or by 64% (150 mg/kg single dose) with negligible effect on body weight (see FIG. 9). Thus, intravenous and oral administrations of apilimod displayed similar efficacy in impairing the growth of SU-DHL-6 tumors in vivo.

The second arm of the study evaluated efficacy of apilimod when combined with ibrutinib in the same SUDHL-6 human DLBCL cancer xenograft model using the same protocol as described above. Each mouse was inoculated subcutaneously in the right flank with SU-DHL-6 tumor cells (5×10⁶) in 0.1 ml of PBS with Matrigel (1:1). The tumors were then grown to a mean size of approximately 80-120 mm³ and the mice were then split into 6 groups and treated as detailed in the Table 6.

TABLE 6
SUDHL-6 cell line xenograft experiment

| | | | | Administration | Number |
|---|---|---|---|---|---|
| Group | Treatment | Dose | Dosing schedule | route | of mice |
| 1 | Vehicle | NA | QD × 5-2 days off- | p.o. + i.v. | 6 |
| | | | QD × 5 | | |
| 2 | Apilimod Free Base | 75 mg/kg | QD × 5-2 days off- | p.o. | 6 |
| | | | QD × 5 | | |
| 3 | Ibrutinib | 10 mg/kg | QD × 12 | i.v. | 6 |
| 4 | Ibrutinib | 20 mg/kg | QD × 12 | i.v. | 6 |
| 5 | Apilimod Free Base + | 75 mg/kg + | QD × 5-2 days off- | p.o. + i.v. | 6 |
| | Ibrutinib | 10 mg/kg | QD × 5 + QD × 12 | | |
| 6 | Apilimod Free Base + | 75 mg/kg + | QD × 5-2 days off- | p.o. + i.v. | 6 |
| | Ibrutinib | 20 mg/kg | QD × 5 + QD × 12 | | |

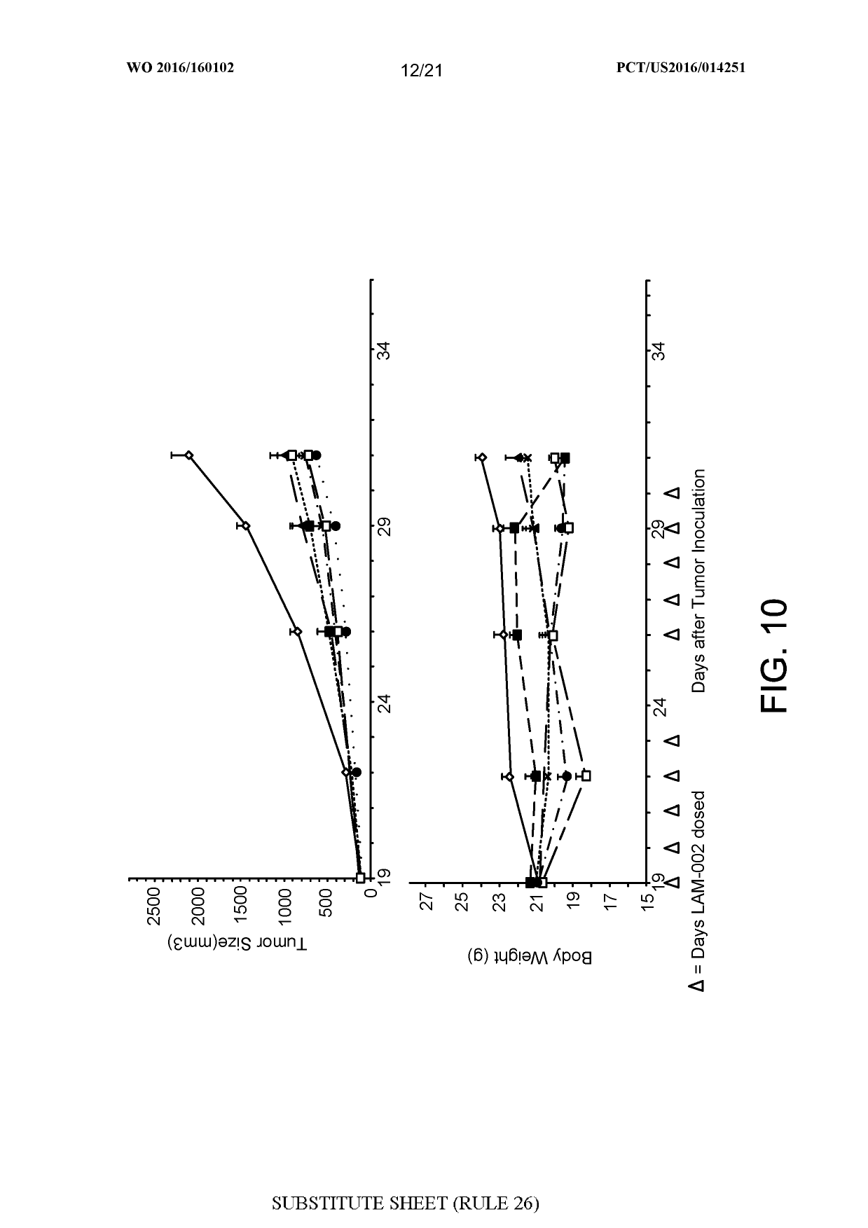

Tumor size was measured twice a week in two dimensions using a caliper, and the volume is expressed in mm³ using the formula: V=0.5 a×b² where a and b are the long and short diameters of the tumor, respectively. The mice were monitored for 31 days and significant growth inhibition was observed in the 75 mg/kg apilimod (57%), 10 mg/kg ibrutinib (54%), and 20 mg/kg ibrutinib (64%) treatment arms. The combination of 75 mg/kg apilimod with ibrutinib further reduced tumor growth in a dose dependent manner; 10 mg/kg ibrutinib (65%) and 20 mg/kg ibrutinib (70%)(see FIG. 10).

### Example 6: Apilimod is a Highly Selective Binder of PIKfyve Kinase

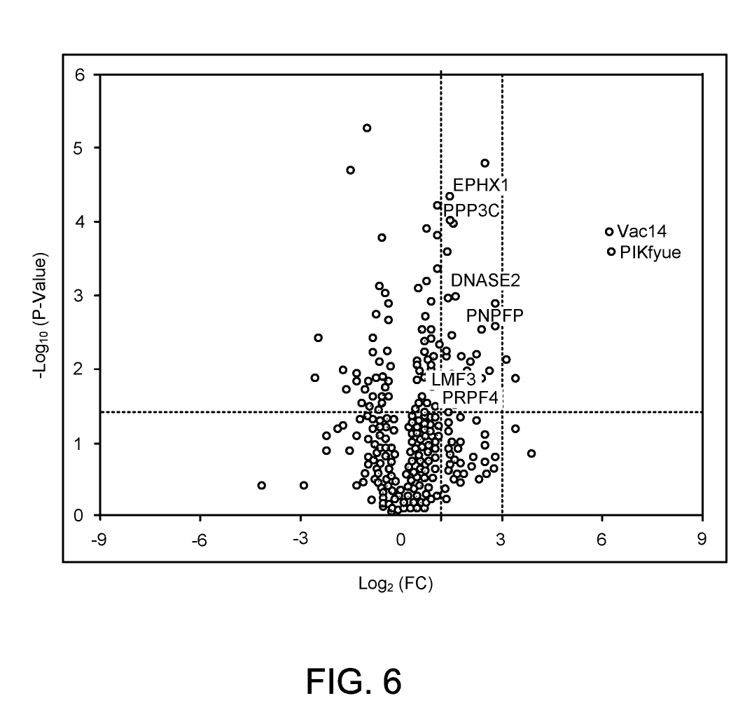

In order to identify the cellular target of apilimod in cancer cells, whole cell lysate prepared from human neuroglioma cells was used to identify its binding partners using chemical capture mass spectrometry (CCMS). This work was performed at Caprotec Bioanalytics GmbH, Berlin Germany. See Michaelis et al., J. Med. Chem., 55 3934-44 (2012) and references cited therein. Briefly, two capture compound variants employing apilimod as selectivity function attached in a single orientation were synthesized and analyzed by LC-MS and 1H-NMR to ensure identity and purity. Capture conditions were optimized in whole cell lysate, e.g. minimization of non-specific interactions of the proteins with capture compounds, concentration of reagents and proteins to obtain maximum binding of proteins and capture compounds, etc. One capture compound was selected to identify specific protein binders in the CCMS experiments using apilimod as a competitor ligand. Proteins that are detected by LC-S in the capture assay and that are significantly diminished in competition control experiments are considered to be specific binders. These specific binders were further subjected to stringent data analysis criteria to determine specificity after unbiased data evaluation. Specific protein binders were ranked according to their fold change (FC) values in the capture experiments. Only two proteins were identified as high probability candidate target proteins of apilimod: PIKfyve and Vac14 (see FIG. 6). FC and p-values for these proteins in the four different capture compound concentration experiments are shown in Table 7.

TABLE 7
Capture Compound
Concentrations

| | | 0.1 μM | 0.5 μM | 1.0 μM | 2.0 μM |
|---|---|---|---|---|---|
| PIKfyve | log₂(FC) | 6.3 | 6.2 | 4.1 | 4.3 |
| | −log₁₀(p-value) | 3.7 | 2.8 | 5.1 | 3.9 |
| Vac14 | log₂(FC) | 6.2 | 5.6 | Inf. | 3.9 |
| | −log₁₀(p-value) | 3.9 | 3.8 | 1.9 | 3.6 |

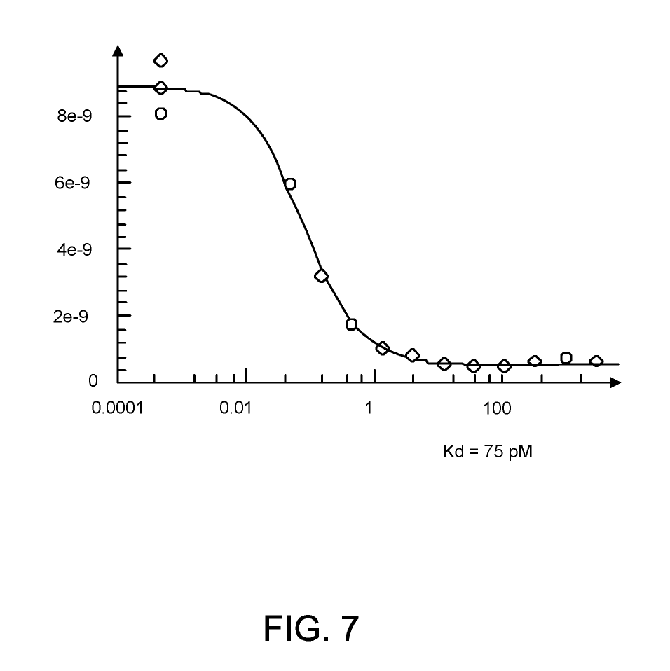

In a separate study, protein kinase profiling of apilimod was conducted to identify kinase targets (DiscoveRx, Fremont, Calif.). A dissociation constant (K_{d}) study was performed using apilimod at increasing concentrations (0.05-3000 nM) against PIKfyve, a known target of apilimod. The experiment was performed in duplicate and the K_{d} was determined to be 0.075 nM (range 0.069-0.081 nM) (FIG. 7).

Next, apilimod was screened against a comprehensive panel of kinases (PIKfyve not included). In total, 456 kinases, including disease-relevant kinases, were assayed for their ability to bind with apilimod. The screening concentration of apilimod was 1 μM, a concentration that is >10,000 times greater than the K_{d} for apilimod against PIKfyve. The results from the screen showed that apilimod did not bind to any of the 456 kinases tested.

Together, these results demonstrate that apilimod binds with high selectivity in cancer cells to a single cellular kinase, PIKfyve. PIKfyve is an enzyme that binds to PI(3)P and catalyzes the formation of the lipid second messengers PI(3,5)P2 and PI(5)P and others have shown that apilimod is also a potent and specific inhibitor of this kinase PIKfyve in normal cells. Cai X et al., Chem Biol. 2013 Jul. 25; 20(7):912-21. As discussed in more detail below, in order to understand the mechanism of apilimod's selective cytotoxicity against cancer cells, we conducted a series of experiments aimed at elucidating its biological activity in cancer cells.

### Example 7: Mechanism of Anti-Cancer Activity of Apilimod

Apilimod is known to be a potent inhibitor of the inflammatory cytokines IL-12 and IL-23. To the extent apilimod was indicated for treating a disease or disorder, it was predicated on this activity. Although the clinical testing of apilimod focused on its potential efficacy in autoimmune and inflammatory diseases such as psoriasis, rheumatoid arthritis, and Crohn's disease, there were a few published suggestions that apilimod might be useful against cancers, and specifically against cancers in which c-rel or IL-12/23 were acting as pro-proliferative factors. See e.g., WO 2006/128129 and Baird et al., Frontiers in Oncology 3:1 (2013), respectively. Surprisingly, and contrary to these expectations predicated on apilimod's IL-12/23 inhibitory activity, we found no correlation between any of c-Rel expression (c-Rel is a transcription factor for the IL-12/23 genes), IL-12, or IL-23 expression and sensitivity to apilimod in the tested cell lines (data not shown).

Briefly, gene expression data from the Cancer Cell Line Encyclopedia (CCLE) was analyzed for the 22 B cell lymphoma lines for which we obtained dose response curves against apilimod (see Table 8).

TABLE 8
22 B Cell Lymphoma Lines analyzed for gene expression and response to apilimod.
Epstein Barr status and nuclear cREL status is noted. ND = No Data

| | | | IC50 | | Nuclear |
|---|---|---|---|---|---|
| Number | B Cell Lymphoma Model | Cell Line | (nM) | EBV | REL |
| 1 | Human Burkitt's lymphoma | ST486 | 25 | No | ND |
| 2 | Human Burkitt's lymphoma | Daudi | 200 | Yes | Yes |
| 3 | Human Burkitt's lymphoma | EB1 | 174 | Yes | ND |
| 4 | Human Burkitt's lymphoma | GA-10 | 382 | No | ND |
| 5 | HumanMantle Cell Lymphoma | Rec-1 | 300 | No | ND |
| 6 | Human Mantle Cell Lymphoma | JeKo-1 | 70 | No | ND |
| 7 | Human Diffuse Large B Cell Lymphoma-GCB | SUDHL-4 | 25 | No | Yes |
| 8 | Human Diffuse Large B Cell Lymphoma-GCB | SUDHL-6 | 80 | No | ND |
| 9 | Human Diffuse Large B Cell Lymphoma-GCB | DB | 150 | No | ND |
| 10 | Human Diffuse Large B Cell Lymphoma-GCB | Toledo | 270 | No | ND |
| 11 | Human Diffuse Large B Cell Lymphoma-GCB | SUDHL-10 | 20 | No | ND |
| 12 | Human Diffuse Large B Cell Lymphoma-GCB | WSU-DLCL2 | 160 | No | ND |
| 13 | Human Diffuse Large B Cell Lymphoma-GCB | OCI-Ly19 | 380 | Yes | ND |
| 14 | Human Burkitt's lymphoma | Namalwa | 600 | Yes | ND |
| 15 | Human Burkitt's lymphoma | CA46 | >10,000 | No | ND |
| 16 | Human Burkitt's lymphoma | Raji | >10,000 | Yes | Yes |
| 17 | Human Mantle Cell Lymphoma | GRANTA-519 | >10,000 | Yes | ND |
| 18 | Human Follicular B Cell Lymphoma | RL | >10,000 | ND | ND |
| 19 | Human Follicular Lymphoma-DLBCL-GCB | DOHH-2 | 700 | No | ND |
| 20 | Human Diffuse Large B Cell Lymphoma-GCB | HT | 642 | No | ND |
| 21 | Human Diffuse Large B Cell Lymphoma-GCB | Pfeiffer | 2,620 | ND | ND |
| 22 | Human Diffuse Large B Cell Lymphoma-GCB | KARPAS-422 | >10,000 | No | ND |

Expression of c-REL was compared in sensitive (IC₅₀ less than 500 nM) and insensitive (IC₅₀ greater than 500 nM) lines by unpaired t-test. No statistically significant relationship between c-REL expression and sensitivity was found (p=0.97). Furthermore, no detection of a significant relationship between sensitivity to apilimod and either the presence of constitutive nuclear c-REL or infection with Epstein Barr virus in cell lines for which data has been published was found. The cell lines tested included the following apilimod sensitive (#1-13) and insensitive (#14-22) B cell lymphoma lines: Human Burkitt's lymphoma cell lines 1-4 (ST486, Daudi, EB1, GA-10), Human Mantle Cell Lymphoma 5-6 (Rec-1, JeKo-1), Human Diffuse Large B Cell Lymphoma-GCB 7-13 (SUDHL-4, SUDHL-6, DB, Toledo, SUDHL-10, WSU-DLCL2, OC1-Ly19), Human Burkitt's Lymphoma 14-16 (Namalwa, CA46, Raji), Human Mantle Cell Lymphoma 17 (GRANTA-519), Human Follicular B Cell Lymphoma 18 (RL), Human Follicular Lymphoma-DLBCL-GCB 19 (DOHH-2), Human Diffuse Large B Cell Lymphoma-GCB (HT, Pfeiffer, KARPAS-422).

The expression of IL-12A, IL-12RB1, IL-12RB2, IL-12B, IL-23A and IL-23R was further analyzed in a diverse group of 75 cancer cell lines, including the aforementioned 22 lymphoma lines (see Table 9).

TABLE 9
Various Cancer cell lines

| | | | IC50 |
|---|---|---|---|
| Number | Cancer Model | Cell Line | (nM) |
| 1 | Human Burkitt's lymphoma | ST486 | 25 |
| 2 | Human Mantle Cell Lymphoma | JeKo-1 | 70 |
| 3 | Human Diffuse Large B Cell Lymphoma-GCB | SUDHL-4 | 25 |
| 4 | Human Diffuse Large B Cell Lymphoma-GCB | SUDHL-6 | 80 |
| 5 | Human Burkitt's lymphoma | Daudi | 200 |
| 6 | Human histiocytic lymphoma | U937 | 106 |
| 7 | Human lung carcinoma | A549 | 110 |
| 8 | Human colorectal cancer | HCT116 | 125 |
| 9 | Human B-cell lymphoma | DB | 150 |
| 10 | Human Diffuse Large B Cell Lymphoma-GCB | WSU-DLCL2 | 160 |
| 11 | Human Colorectal | HCT-15 | 200 |
| 12 | Human Colorectal | SW480 | 90 |
| 13 | Human Colorectal | COLO-205 | 380 |
| 14 | Human Colorectal | SW620 | 90 |
| 15 | Human T-cell leukemia | Jurkat | 200 |
| 16 | Human neuroglioma | H4 | 250 |
| 17 | Human Diffuse Large B Cell Lymphoma-GCB | Toledo | 270 |
| 18 | Human B cell Non-Hodgkin's Lymphoma | Rec-1 | 300 |
| 19 | Human Hodgkin's lymphoma | KMH-2 | 181 |
| 20 | Human Burkitt's lymphoma | EB1 | 174 |
| 21 | Human Diffuse Large B Cell Lymphoma-GCB | SUDHL-10 | 20 |
| 22 | Human Burkitt's lymphoma | GA-10 | 382 |
| 23 | Human Diffuse Large B Cell Lymphoma-GCB | OCI-Ly19 | 380 |
| 24 | Human Diffuse Large B Cell Lymphoma-GCB | HT | 642 |
| 25 | Human Diffuse Large B Cell Lymphoma-GCB | Pfeiffer | 2,620 |
| 26 | Human Burkitt's lymphoma | Namalwa | 600 |
| 27 | Human Follicular B Cell Lymphoma-GCB | DOHH-2 | 700 |
| 28 | Human Bladder carcinoma (GATOR −/−) | SW780 | 1000 |
| 29 | Human colorectal cancer | MDST8 | 1000 |
| 30 | Human Burkitt's lymphoma | Raji | 10,000 |
| 31 | Human Hodgkin's lymphoma | HD-MyZ | >1000 |
| 32 | Human Hodgkin's lymphoma | L540 | >1000 |
| 33 | Human Hodgkin's lymphoma | HDLM-2 | >1000 |
| 34 | Human Burkitt's lymphoma | CA46 | >10,000 |
| 35 | Human Anaplastic Large Cell Lymphoma | SUDHL-1 | 590 |
| 36 | Human lung carcinoma | H1734 | 1500 |
| 37 | Human colorectal cancer | SW1116 | 1500 |
| 38 | Human Colorectal | COLO-320DM | 2,060 |
| 39 | Human neuroblastoma | A172 | 2000 |
| 40 | Human lung carcinoma | H1693 | 2000 |
| 41 | Human lung carcinoma | H460 | >2000 |
| 42 | Human lung carcinoma | H358 | >2000 |
| 43 | Human pancreatic cancer | CAPAN2 | >2000 |
| 44 | Human pancreatic cancer | PANC1 | >2000 |
| 45 | Human pancreatic cancer | MiaPaCa-2 | >2000 |
| 46 | Human pancreatic cancer | AsPC1 | >2000 |
| 47 | Human prostate cancer | DU145 | >2000 |
| 48 | Human acute myelogenous leukemia | KG-1 | >2500 |
| 49 | Human prostate cancer | LnCap | 3000 |
| 50 | Human T-cell lymphoma | HH | 3,300 |
| 51 | Human T-cell leukemia | MOLT-4 | 3,300 |
| 52 | Human prostate cancer | 22RV1 | >5000 |
| 53 | Human colorectal cancer | DLD-1 | >5000 |
| 54 | Human myelogenous leukemia | K562 | >5000 |
| 55 | Human colorectal cancer | RKO | >5000 |
| 56 | Human ovarian | TOV-21G | 7000 |
| 57 | Human prostate cancer | PC-3 | 10,000 |
| 58 | Human Hodgkin's lymphoma | L428 | 10,000 |
| 59 | Human plasmacytoma | RPMI-8226 | >10,000 |
| 60 | Human lung carcinoma | NCI-1975 | >10,000 |
| 61 | Human breast cancer | CAMA1 | >10,000 |
| 62 | Human neuroblastoma | SW1088 | >10,000 |
| 63 | Human neuroblastoma | M0591K | >10,000 |
| 64 | Human neuroblastoma | U-118 MG | >10,000 |
| 65 | Human neuroblastoma | U-87 MG | >10,000 |
| 66 | Human acute monocytic leukemia | THP1 | >10,000 |
| 67 | Human Diffuse Large B Cell Lymphoma-GCB | KARPAS-422 | >10,000 |
| 68 | Human Follicular B Cell Lymphoma | RL | >10,000 |
| 69 | Human Mantle Cell Lymphoma | GRANTA-519 | >10,000 |
| 70 | Human bronchioalveolar | NCI-H1650 | >20,000 |
| 71 | Human bronchioalveolar | SW1573 | >20,000 |
| 72 | Human bronchioalveolar | NCI-H1781 | >20,000 |
| 73 | Human bronchioalveolar | NCI-H1666 | 20,000 |
| 74 | Human Colorectal | LOVO | >10,000 |
| 75 | Human Colorectal | HT-29 | >10,000 |

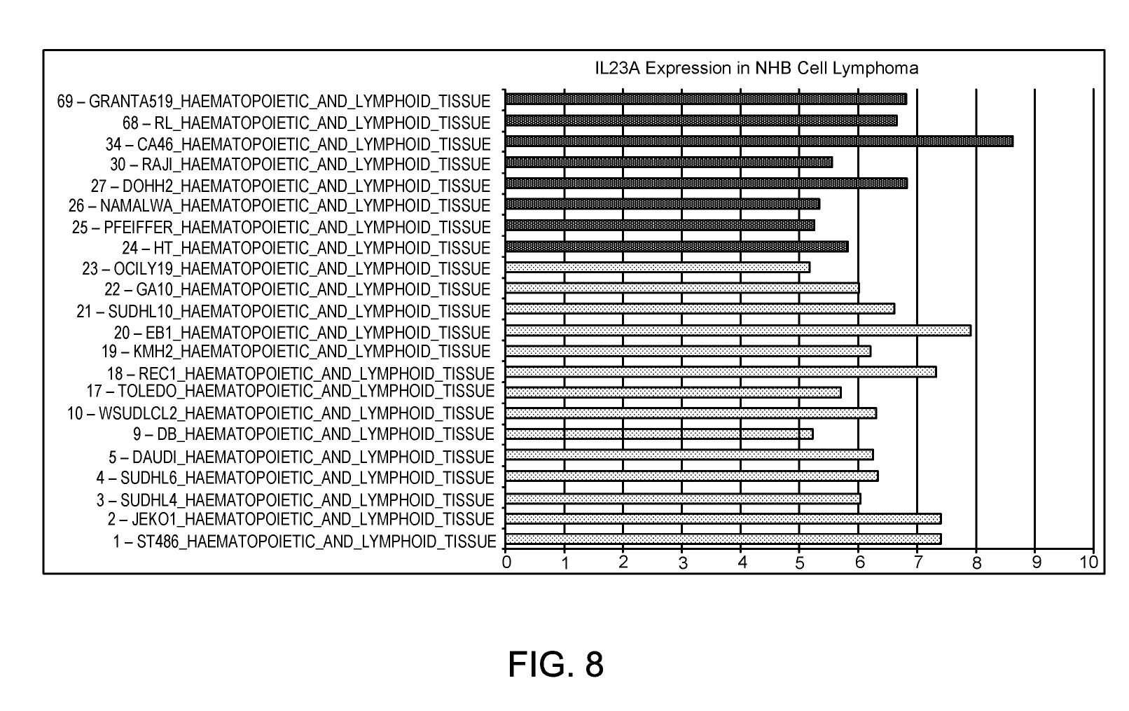

Briefly, gene expression data from the CCLE was analyzed for the 75 cancer cell lines for which dose response curves against apilimod were obtained. The expression of each interleukin gene was compared in sensitive (IC₅₀ less than 500 nM) and insensitive (IC₅₀ greater than 500 nM) lines by unpaired t-test. No statistically significant relationship was found with the sole exception of IL-23A (p=0.022). IL-23A has been previously noted to be elevated in apilimod sensitive non small cell lung cancer lines, and recombinant IL-23A was noted to increase proliferation of non small cell lung cancer lines (see Baird et al. 2013, supra). Importantly, the statistical significance of IL-23A expression in sensitive cancer lines appears to be driven entirely by just two colon cancer lines. Furthermore IL-23A expression is not a statistically significant predictor of sensitivity in Non-Hodgkin's B cell lymphoma (FIG. 8). Global gene expression data from the CCLE database was analyzed for a reliable two gene biomarker for apilimod sensitivity in the 22 B cell lymphoma lines.

Additional experiments demonstrated that apilimod's cytotoxic activity was based at least in part on its inducing cellular apoptosis. Apoptosis was quantified and distinguished from necrosis using the Apotox-Glo Triplex assay (Promega, Inc.) according to the manufacturer's instructions. In this assay, viability, apoptosis, and necrosis are assessed simultaneously using three different markers (GF-AFC, Caspase-3/7, and bis-AAF-R110, respectively). FIG. 4 shows apoptotic (middle bar) and necrotic (right bar) markers in apilimod treated in diffuse large B cell lymphoma cells 48 hours after addition of apilimod to the culture media. The left bar shows the viability marker.

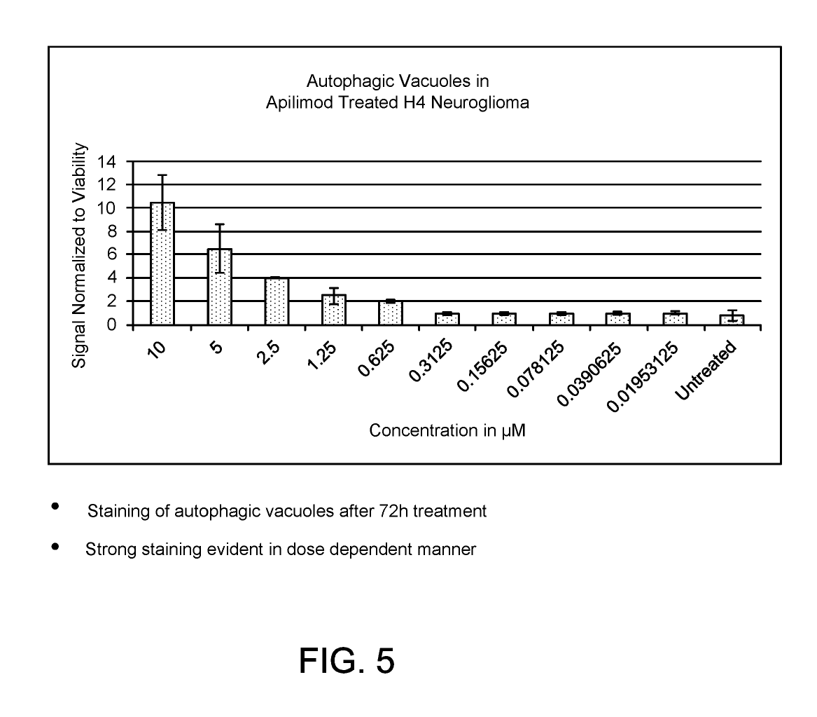

The mechanism of apilimod's cytotoxic activity was further investigated by assaying for autophagic vacuoles after 72 hours of treatment in an H4 neuroglioma cell line (IC₅₀ 250-300 nM). Autophagy was quantified using the Cyto-ID Autophagy detection kit (Enzo) according to manufacturer's directions. FIG. 5 shows that apilimod induced autophagy in a dose-dependent manner.

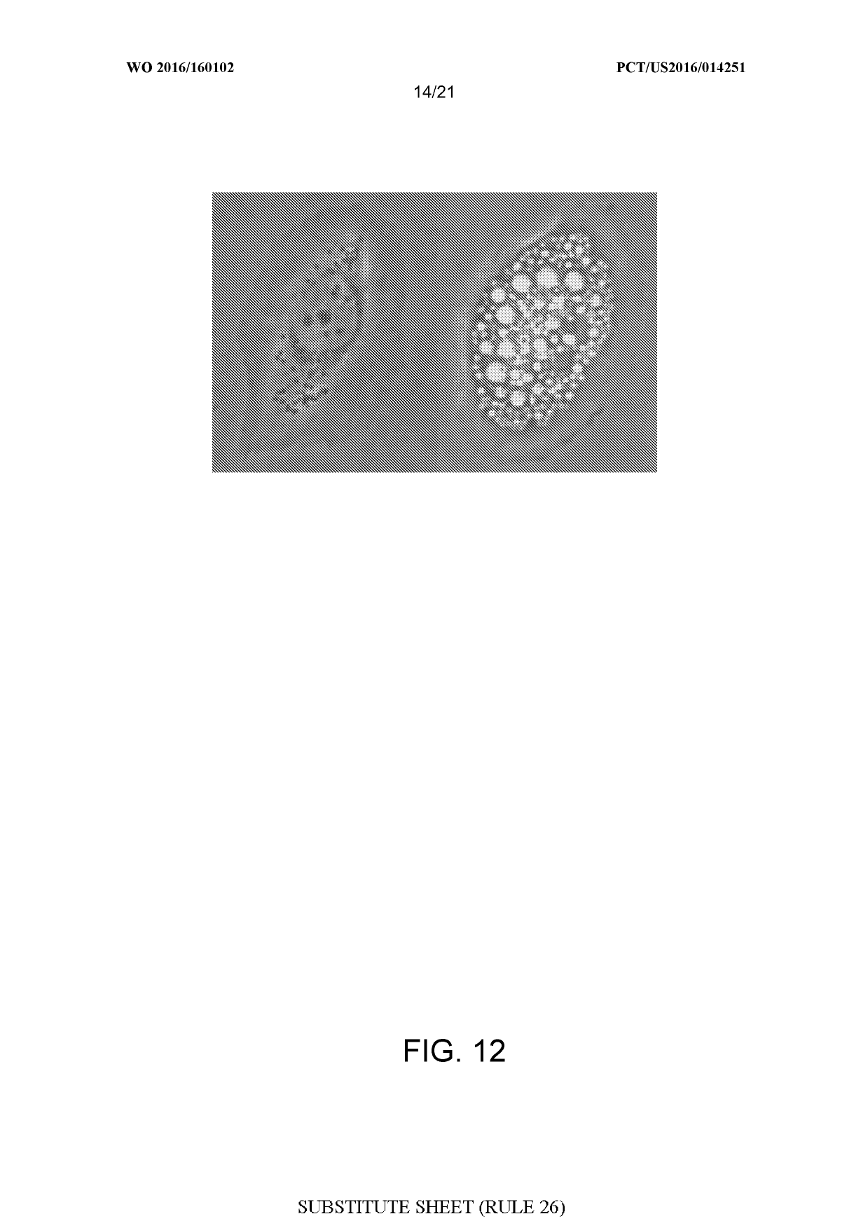

PIKfyve is associated with the cytosolic leaflet of early endosomes and its activity is required for endomembrane homeostasis, endolysosomal function and proper retrograde transport from the endosome to the trans-Golgi network. Introduction of a kinase dead mutant into cells induces a swollen vacuole phenotype that can be rescued by the injection of PI(3,5)P2. Inhibition of PIKfyve by pharmacological methods as well as RNAi also produces swollen vacuoles and disruption of endomembrane dynamics. As shown in FIG. 12, pharmacological disruption of PIKfyve with apilimod induces selective lethality of specific cancer cell lines through disruption of intracellular trafficking.

### Example 8: Synergistic Activity Between Apilimod and Vemurafenib

Yulac614 (resistant to vemurafenib, see Choi et al. 2014) cells were used to conduct drug combination studies to identify synergistic drug pairs. A library of 500 unapproved drugs was used to perform drug screening in the presence or absence of vemurafenib (at IC₂₀=6 μM). The drug library was diluted from 10 mM stock solution to sub-stock solutions of 5, 0.5 and 0.05 mM (1000× final concentration) to give final screening concentrations of 5, 0.5 and 0.05 μM.

30 nL of library drug (at 1000× concentration) was spotted into appropriate wells of a 384 black walled plate (Corning #3712). Duplicate drug-spotted plates were prepared and to them were added Yulac614 cells which were pre-treated with either DMSO (0.01% final) or vemurafenib (6 μM final). The Yulac614 cells were grown in OptiMEM (Life Technologies) containing 5% FBS (Sigma Aldrich F2442-500 mL, Lot 12D370) Penicillin/Streptomycin (100×) (CellGro Ref 30-002). 30 uL of cells were dispensed per well using a Multidrop Combi (Thermo Fisher Scientific) to give a final cell density of 2,000 cells per well.

Plates were incubated for 72 h at 37° C. under an atmosphere of 5% CO₂ in a humidified incubator. Cell viability was determined with CellTiter-Glo® luminescence assay (Promega) as per the manufacturer's instructions. Viability was expressed as a percentage of control (DMSO) cells. The viability of the library drug alone was compared to the library drug+vemurafenib and significant combinations were identified.

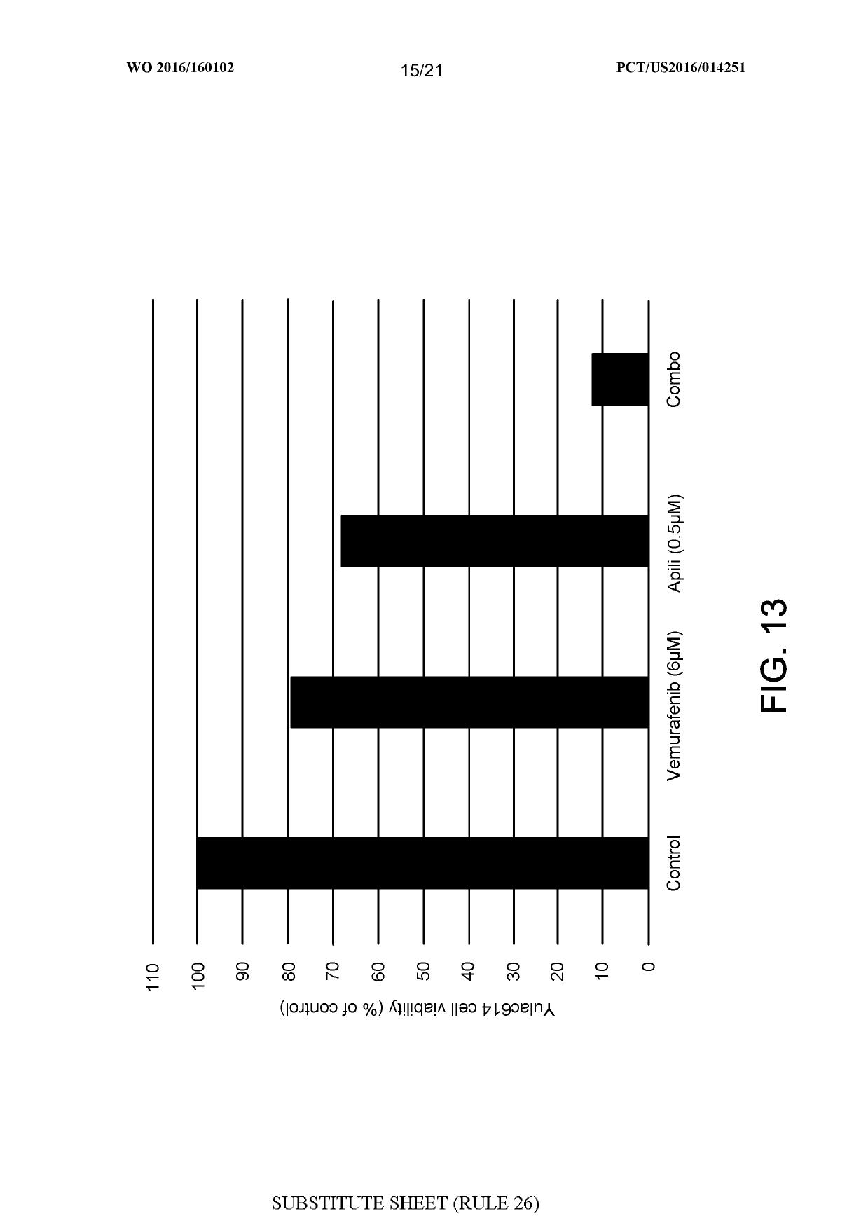

Apilimod was identified as an unapproved drug that in combination with vemurafenib significantly reduced Yulac614 cell viability as compared with either drug alone. See FIG. 13.

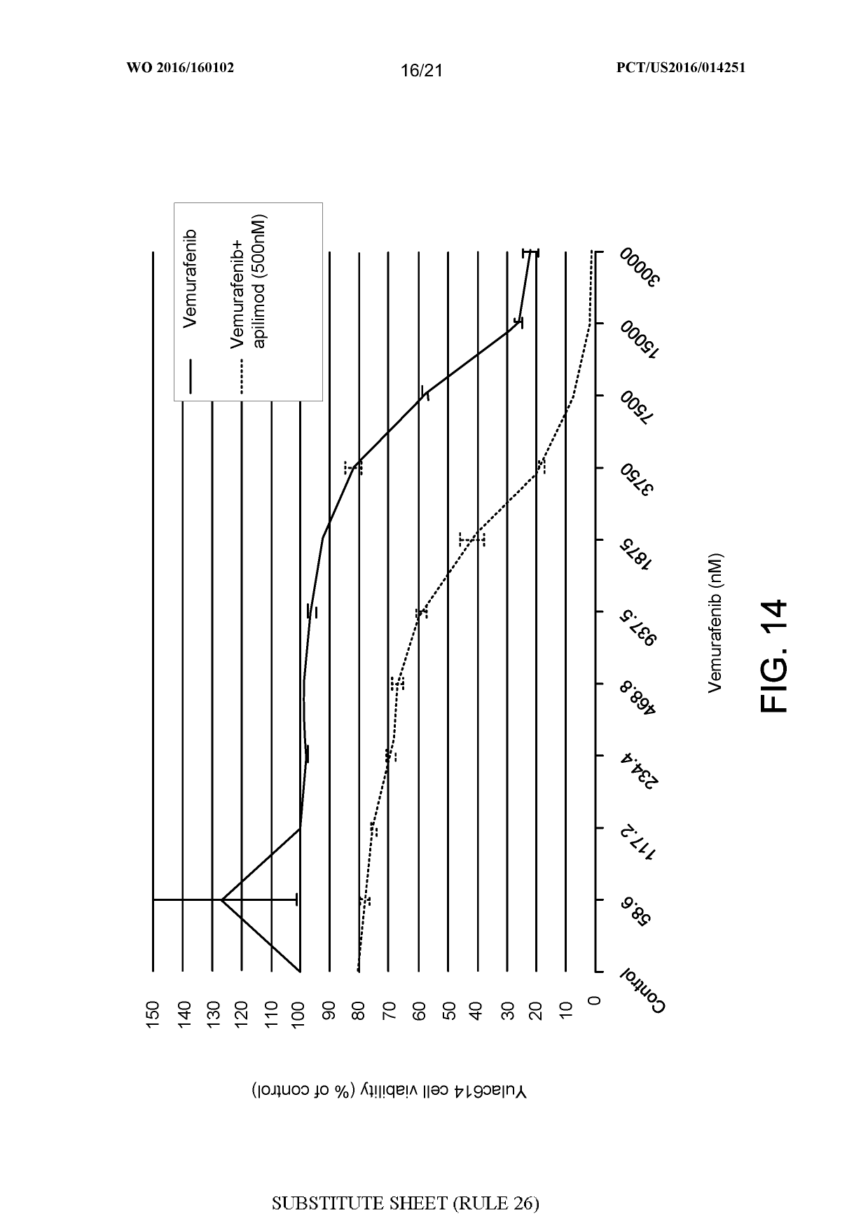

To validate the vemurafenib and apilimod observation, Yulac614 cells were seeded into 96 well plates at a density of 5,000 cells per well in a final volume of 50 uL. Vemurafenib was tested in a 10 point-concentration response curve (58.6-30,000 nM; 2-fold dilutions) in the presence and absence of apilimod (at IC₂₀=500 nM). 50 uL of the 10 point drug dilution series (at 2× concentration) was added to the cells. Plates were incubated at 37° C. under an atmosphere of 5% CO₂ in a humidified incubator. 72 hours after compound addition relative cell viability was determined by CellTiter-Glo® luminescence assay (Promega) as per the manufacturer's instructions, and values were expressed as a percentage relative to vehicle (DMSO) treated control cells (set to 100%). FIG. 14 depicts a 10 point concentration response curve of vemurafenib (58.6-30,000 nM) alone (black line) or with apilimod (500 nM) (grey line). The results demonstrate that apilimod in combination with vemurafenib is more effective than vemurafenib alone in reducing cell viability of Yulac614 cells.

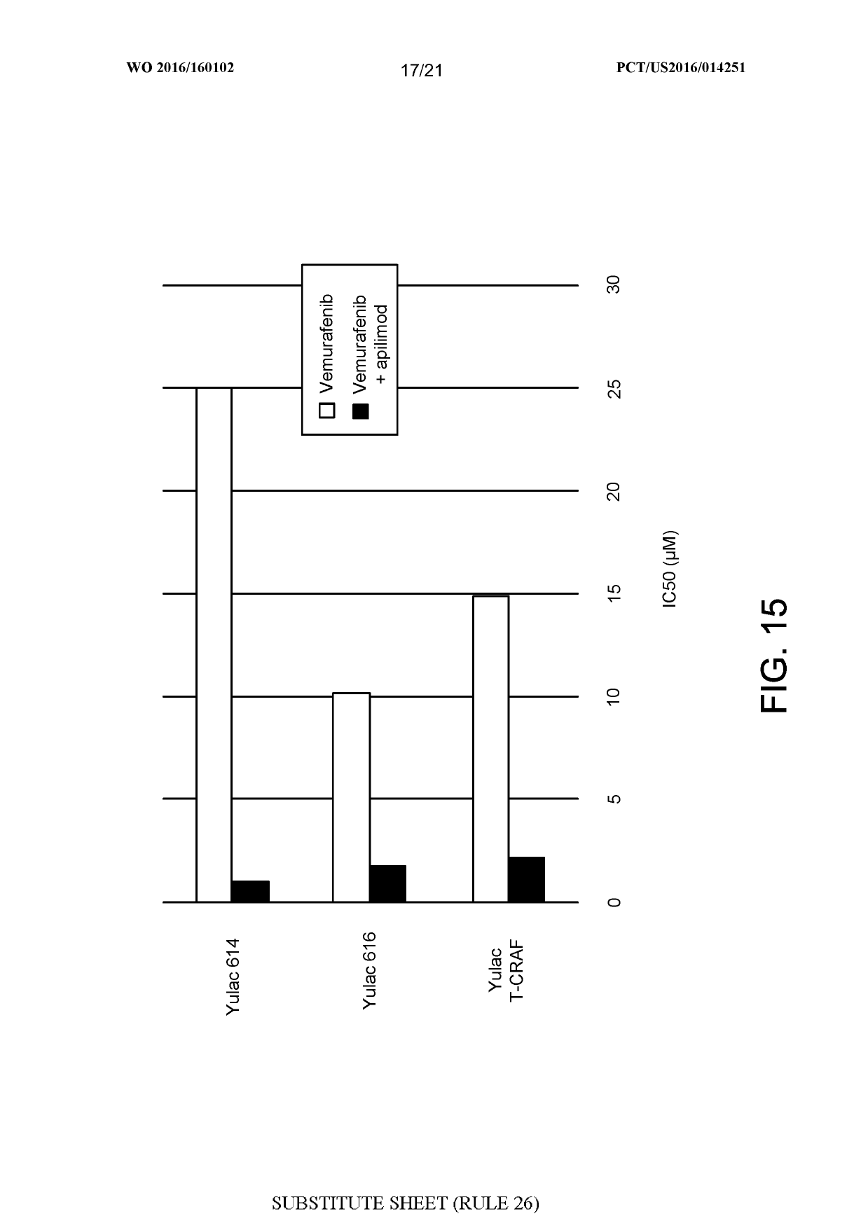

In another experiment, Yulac (parental-sensitive to vemurafenib), Yulac 614, Yulac 616, Yulac T-CRAF (all resistant to vemurafenib), see Choi et al. 2014) were grown in OptiMEM (Life Technologies) containing 5% FBS (Sigma Aldrich F2442-500 mL, Lot 12D370) Penicillin/Streptomycin (100×) (CellGro Ref 30-002). For combination studies, cells were seeded at a density of 5000 cells per well into 96 well plates in a final volume of 50 μL. Cells were treated with apilimod alone (final concentration 13.7-30000 nM; 3-fold dilutions and a total of 8 dilutions), with vemurafenib alone (final concentration 13.7-30000 nM; 3-fold dilutions and a total of 8 dilutions) or the combination of each concentration of apilimod with each concentration of vemurafenib (8×8 matrix). Cells were treated for 72 h before proliferation was assessed using CellTiterGlo® (Promega). For calculation of synergy, CalcuSyn (version 2.11, Biosoft) was used to determine the combination index (CI) as defined by Chou et al. (Chou T C, Talalay P. Quantitative analysis of dose-effect relationships: the combined effects of multiple drugs or enzyme inhibitors. Adv Enzyme Regul 1984; 22:27-55). Thus, drug combinations producing CI values >1 are antagonistic, CI=1 are additive and CI<1 are synergistic. Using this approach, apilimod was found to act synergistically with vemurafenib in each of these cell lines (see Table 10). The change in sensitivity to vemurafenib by apilimod was determined using GraphPad Prism4 software to calculate IC50 values. As shown in FIG. 15, vemurafenib in combination with apilimod at 370 nM reduced the IC50 by 5.5 to 25 fold compared to vemurafenib alone.

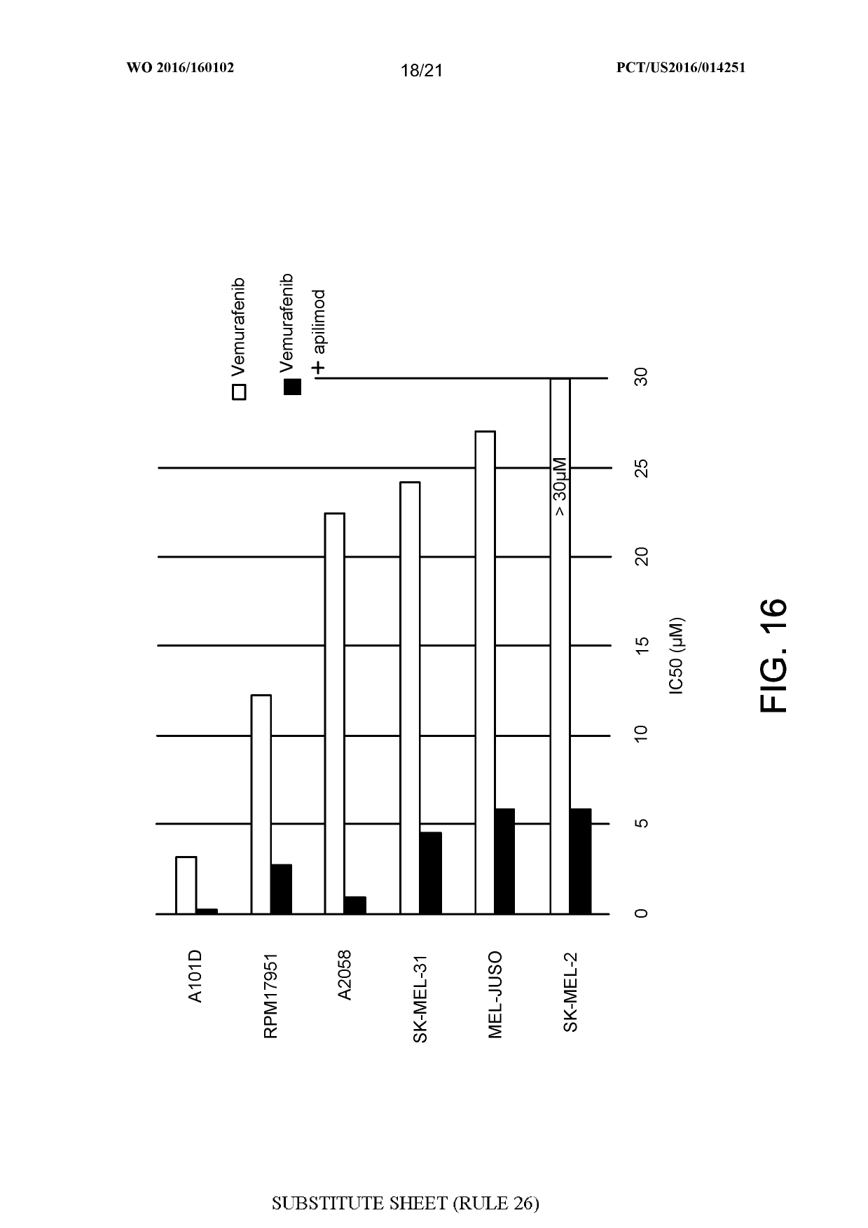

To extend these findings, a panel of melanoma cell lines that displayed inherent resistance to vemurafenib was chosen for further study with apilimod. A101D, SK-MEL-2, SK-MEL-31, RPMI7951, A2058 (grown in DMEM supplemented with 10% fetal bovine serum) and MEL-JUSO (grown in RPMI supplemented with 10% fetal bovine serum) cells were obtained from ATCC. Cells were seeded at optimal density in 96 well plates in a final volume of 50 μL. Cells were treated with apilimod alone (final 78.1-10000 nM; 2-fold dilutions and a total of 8 dilutions), with vemurafenib alone (final concentration 234-30000 nM; 2-fold dilutions and a total of 8 dilutions) or the combination of each concentration of apilimod with each concentration of vemurafenib (8×8 matrix). Cells were treated for 72 h before proliferation was assessed using CellTiterGlo® (Promega). For calculation of synergy, CalcuSyn (version 2.11, Biosoft) was used to determine the combination index (CI) as defined by Chou et al. (Chou T C, Talalay P. Quantitative analysis of dose-effect relationships: the combined effects of multiple drugs or enzyme inhibitors. Adv Enzyme Regul 1984; 22:27-55). Thus, drug combinations producing CI values >1 are antagonistic, CI=1 are additive and CI<1 are synergistic. Using this approach, apilimod was found to act synergistically with vemurafenib in each cell line (see Table 11). The change in sensitivity to vemurafenib by apilimod was determined using GraphPad Prism4 software to calculate IC50 values. As shown in FIG. 16, vemurafenib in combination with apilimod at 185 nM (A101D, RPMI7951, A2058 and MEL-JUSO cells) or 312 nM (SK-MEL-2 and SK-MEL-31) reduced the IC50 by 4.5 to 25 fold compared to vemurafenib alone.

### Example 9: Prediction of In Vivo Anti-Ebola Activity in Humans

Inhibition of cancer cell proliferation and inhibition of Ebola virus infection share a common mechanism i.e. inhibition of PIKfyve leading to vacuole formation and loss of intracellular trafficking. In the above clinical study, the trough TAEC values were greater than 25 mg/mL (60 nM). Since vacuole formation in cells at apilimod concentrations as low as 20 nM have been observed, one could conclude that oral administration of apilimod free base at doses ranging from 70 to 1000 mg/day should provide continuous PIKfyve inhibition to maintain vacuolization of cells and block Ebola infection in clinical therapy in patients.

Furthermore, it was shown in female Balb/c mice, a strain often used for in vivo Ebola infection studies, that constant infusion of apilimod as the bis-mesylate salt, using subcutaneously implanted osmotic mini-pumps (e.g. Alzet models 1007D, 15 mg/kg/day; model 2001, 30 mg/kg/day; vehicle: 25% DMSO, 25% Cremaphor, 50% sterile water) can provide sustained blood concentrations of apilimod in excess of 0.5 μM and 1 μM, respectively, as measured at 24 h (Table 12). Apilimod bis-mesylate was well tolerated under these conditions, with no visible adverse effects. In contrast, when apilimod was administered by intraperitoneal injection (30 mg/kg in 0.5%, methylcellulose in water) the blood concentration was below the level of quantitation at the 24 h timepoint.

Therefore, intravenous infusion of apilimod bis-mesylate to humans in an appropriate formulation (highly water soluble) at an appropriate rate, is expected provide potent Ebola virucidal activity and may be useful in the acute, critical care setting.

TABLE 12
Comparison of Apilimod plasma concentrations following I.P. injection, continuous S.C.
Infusion, or both simultaneously in Balb/C mice

| | Route | | Vehicle | | Dose (Bis-mesylate salt) | | Apilimod |
|---|---|---|---|---|---|---|---|
| | Pump | I.P. | Pump | I.P. | Pump | I.P. | Ave. Plasma |
| Group | (S.C.) | (injection) | (S.C.) | (injection) | (S.C.) | (injection) | Conc. (μM) |
| 1 | √ | √ | DRW | MC | — | — | — |
| 2 | — | √ | — | MC | — | 30 mg/kg | BQL |
| 3 | √ | √ | DRW | MC | ~15 mg/kg/d | 30 mg/kg | 0.682 |
| 4 | √ | √ | DRW | MC | ~30 mg/kg/d | 30 mg/kg | 1.01 |
| 5 | √ | √ | DRW | DRD | — | 30 mg/kg | — |
| 6 | — | √ | — | DRD | — | 30 mg/kg | BQL |
| 7 | √ | √ | DRW | DRD | ~15 mg/kg/d | 30 mg/kg | 0.613 |
| 8 | √ | √ | DRW | DRD | ~30 mg/kg/d | 30 mg/kg | 1.76 |
| 9 | √ | — | DRW | — | ~15 mg/kg/d | — | 0.755 |
| 10 | √ | — | DRW | — | ~30 mg/kg/d | — | 1.87 |

The following study protocol was carried out to obtain the results discussed above:

### Study Duration

Dosing: Days 1 to 6, PK determination: Day 7

### Formulations

DRW: 25% DMSO, 25% Cremophor RH40, 50% Sterile water.

MC: 0.5% methylcellulose in water.

DRD: 10% DMSO, 13.5% Cremophor RH40, 76.5% 5% dextrose in water.

### Alzet Mini-Pumps

1007D Alzet pump used to dose ˜15 mg/kg/d (Reservoir volume=100 μL)

2001 Alzet pump used to dose ˜30 mg/kg/d (Reservoir volume=200 μL)

Drug concentration is 25 mg/mL in both pumps

### Dosing Volume

10 mL/kg for I.P. injection

0.5 μL/h for 1007D pump

1.0 μL/h for 2001 pump

### Dose Groups

MC formulation with I.P. injection was used in mice in Groups 1 to 4.

DRD formulation with I.P. injection was used in mice in Groups 5 to 8.

DRW formulation was administered by Alzet mini-pump in all groups except 2 and 6. Group 1 and 5 are DRW formulation controls (i.e. no drug).

All groups received I.P. drug or I.P. control except groups 9 and 10—the latter are

the mini-pump infusion ONLY groups.

### Conclusions

1.) S.C. continuous mini-pump infusion of apilimod bis-mesylate, with (Groups 3 and 4, Groups 7 and 8) or without (Groups 9 and 10) concomitant I.P. injection, delivers therapeutically relevant steady state plasma concentrations of apilimod in Balb/c mice (based on Day 7 data).

2.) Steady state plasma concentration is roughly proportional to infusion rate. Since the drug concentration is the same (25 mg/mL) for both infusion rates, steady state plasma concentration is roughly proportional to dose (Group 3 vs 4, Group 7 vs 8, Group 9 vs 10).

3.) I.P. injection alone fails to provide measurable plasma concentration of apilimod 24 h after the final dose, irrespective of formulation (Groups 2 and 6).

4.) S.C. continuous mini-pump infusion alone is sufficient to provide therapeutically relevant steady state plasma concentrations of apilimod.

### Example 10: Several Human Metabolites of Apilimod Potently Inhibit PIKfyve

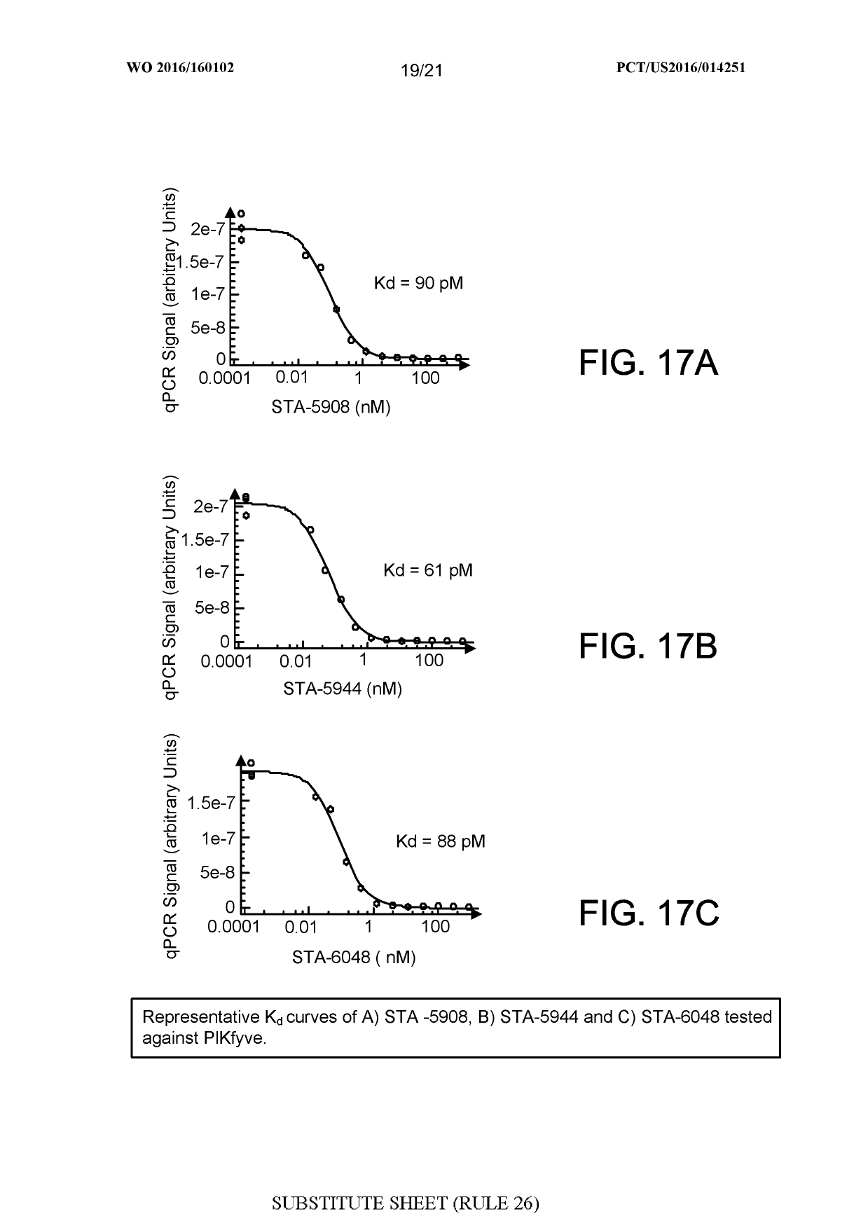

Apilimod has been studied extensively in human clinical trials and many human metabolites have been identified, synthesized chemically and their human pharmacokinetic profile is known. We have discovered unexpectedly that three metabolites of apilimod (STA-5864, STA-5908, and STA-5944) bind to recombinant PIKfyve with similar affinity to apilimod itself. A dissociation constant (K_{d}) study was performed using the 3 compounds at increasing concentrations (0.05-3000 nM) against PIKfyve. The experiment was performed in duplicate and the K_{d} was determined to be 0.09 nM (range 0.092-0.087 nM), 0.061 nM (range 0.06-0.062 nM) and 0.088 nM (range 0.085-0.09) for STA-5908, STA-5944 and STA-6048, respectively (see FIG. 17).

### Example 11: Apilimod Induces Vacuolization and Disrupts Intracellular Trafficking in Cells

Apilimod has been demonstrated to be a potent and specific inhibitor of the phosphoinositide kinase PIKfyve, an enzyme that binds to PI(3)P and catalyzes the formation of the lipid second messengers PI(3,5)P2 and PI(5)P. PIKfyve is associated with the cytosolic leaflet of early endosomes and its activity is required for endomembrane homeostasis, endolysosomal function and proper retrograde transport from the endosome to the trans-Golgi network. Introduction of a kinase dead mutant into cells induces a swollen vacuole phenotype that can be rescued by the injection of PI(3,5)P2. Inhibition of PIKfyve by pharmacological methods as well as RNAi also produces swollen vacuoles and disruption of endomembrane dynamics. It has been discovered that pharmacological disruption of PIKfyve with apilimod induces selective lethality of specific cancer cell lines through disruption of intracellular trafficking (see FIG. 12). Furthermore, it was shown that the metabolites STA-5908, STA-5944 and STA-6048 also cause vacuolation in cells at comparable concentrations.

### Example 12: Human PK Profile of Apilimod and Metabolites

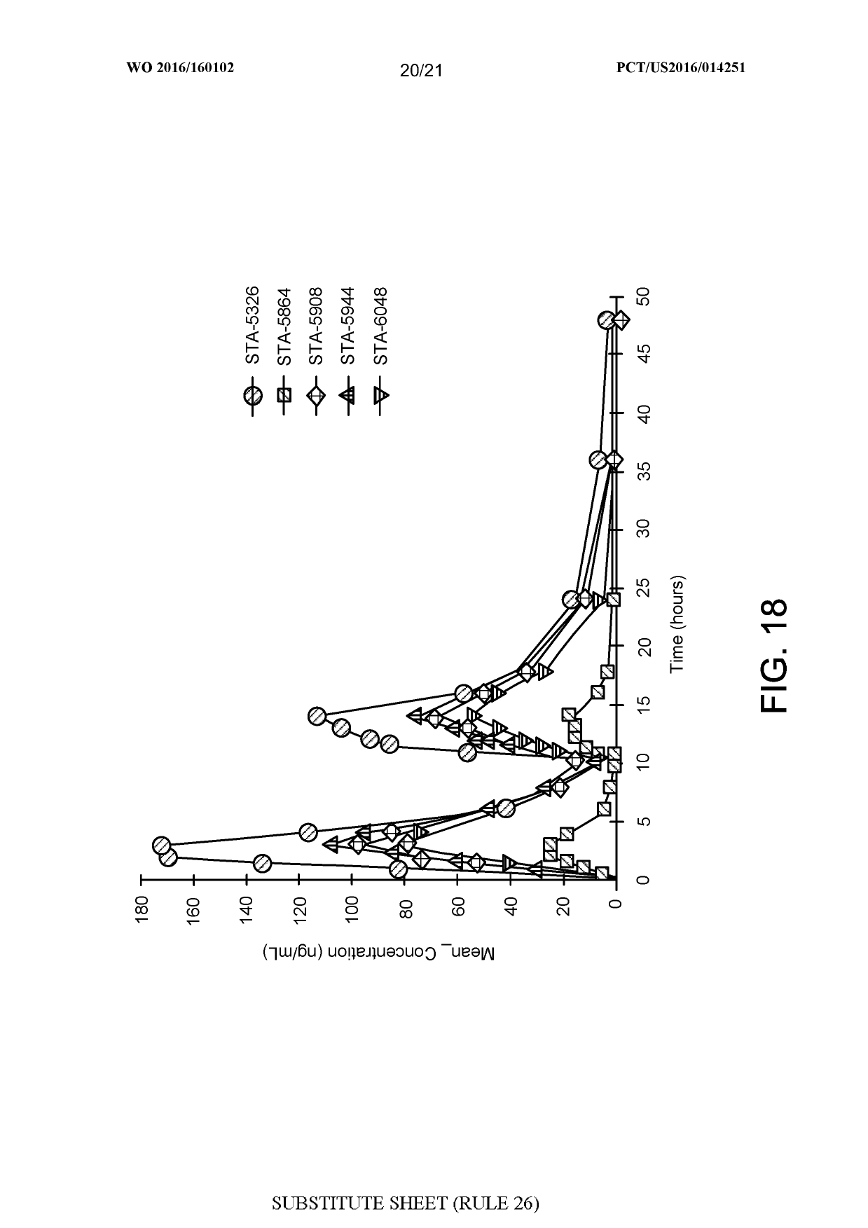

The human plasma profile of apilimod and several metabolites following oral administration (105 mg doses of apilimod free base, given 10 hr apart) is illustrated in FIG. 18. The mean STA-5944 and STA-5908 metabolite profiles are close to those of apilimod, but show a mean Cₘₐₓ that is somewhat less than half of the latter. Both these metabolites are above the IC₅₀ for a significant portion of the 0-24 hr period. As a result of their PIKfyve inhibition and vacuole forming activity, these metabolites add to the pharmacological activity of apilimod in vivo, which has so far been unappreciated.

### Example 13: Quantification of Apilimod Metabolite Effects in Human

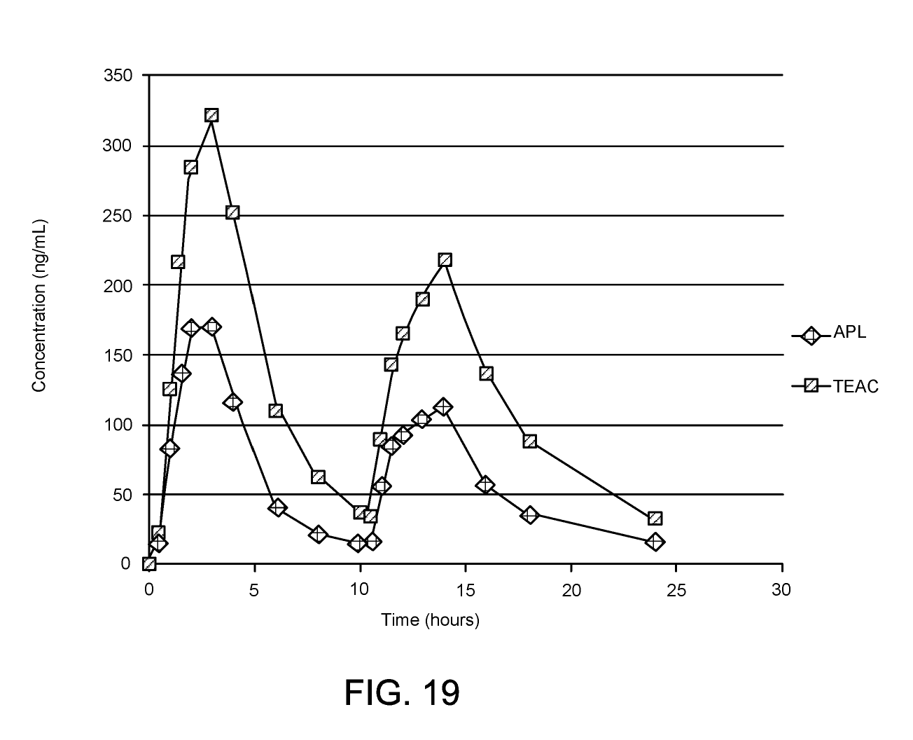

Recent studies have shown that apilimod exhibits potent anti-proliferative activity in cancer cell lines, for the reasons described in Example 3, above. This data can be used to quantitatively adjust the effect of apilimod metabolite concentrations into “Apilimod Equivalent Concentrations” (AEC's) in human clinical subjects. As an example, using data from WSU-DLCL2 cells, and normalizing to apilimod itself, the plasma concentration factors for metabolites STA 5908, STA5944, and STA 6048 were calculated as 0.68, 0.52, and 0.26 respectively. The AEC at each time point in each subject for each metabolite was then summed with the plasma apilimod concentration at that time-point, to give a total AEC (TAEC—see FIG. 19). Thus, the TAEC is substantially higher in humans than would be predicted from apilimod plasma concentrations alone.

1. A method for treating cancer in a human subject in need thereof, the method comprising administering an effective amount of a compound of Formula II to the subject: 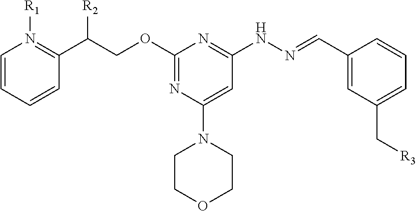
wherein
R₁ is O or absent;
R₂ is H or OH; and
R₃ is H or OH.

2. The method of claim 1, wherein R₂ is OH.

3. The method of claim 1, wherein the effective amount of the compound is the amount effective to inhibit cellular PIKfyve activity in target cells in the subject.

4. The method of claim 1, wherein the cancer is a lymphoma, a melanoma, a renal cancer, or a colon cancer.

5. The method of claim 4, wherein the cancer is a lymphoma or melanoma.

6. The method of claim 5, wherein the cancer is refractory or resistant to standard therapy.

7. The method of claim 5, wherein the cancer is a non-Hodgkins lymphoma.

8. The method of claim 4, wherein the cancer is a renal cancer.

9. The method of claim 8, wherein the renal cancer is refractory or resistant to standard therapy.

10. The method of claim 1, wherein the compound is selected from the group consisting of STA-5864, STA-5944, STA-5908, STA-5919, STA-6035, and STA-6048.

11. The method of claim 10, wherein the compound is in the form a pharmaceutical composition.

12. The method of claim 10, wherein the compound comprises at least 95% or at least 99% enantiomeric excess of the (R)-enantiomer.

13. The method of claim 10, wherein the compound comprises at least 95% or at least 99% enantiomeric excess of the (S)-enantiomer.

14. A pharmaceutical composition comprising a compound of Formula II wherein the compound comprises at least 95% or at least 99% enantiomeric excess of the (R)-enantiomer.

15. A pharmaceutical composition comprising a compound of Formula II wherein the compound comprises at least 95% or at least 99% enantiomeric excess of the (S)-enantiomer.

16. The pharmaceutical composition of claim 14, wherein the compound is selected from the group consisting of STA-5864, STA-5944, STA-5908 STA-5919, STA-6035, and STA-6048.

17. (canceled)

18. A method for inhibiting cellular PIKfyve activity in a target cell, the method comprising contacting the target cell with an amount of a compound of Formula II effective to inhibit PIKfyve activity in the cell: 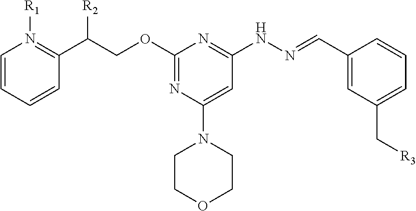
wherein
R₁ is O or absent;
R₂ is H or OH; and
R₃ is H or OH.

19. The method of claim 18, wherein R₂ is OH.

20. The method of claim 18, wherein the compound is selected from the group consisting of STA-5864, STA-5944, STA-5908, STA-5919, STA-6035, and STA-6048.

21. (canceled)

22. (canceled)

23. (canceled)
